(19) United States
(12) Patent Application Publication
(10) Pub. No.: US 2019/0264264 A1
(43) Pub. Date: Aug. 29, 2019

(54) SYSTEMS AND METHODS FOR ANALYZING RNA TRANSCRIPTS

(71) Applicant: integrated Nano-Technologies, Inc., Henrietta, NY (US)

(72) Inventors: Dennis M. Connolly, Rochester, NY (US); Richard S. Murante, Rochester, NY (US); Nathaniel E. Wescott, West Henrietta, NY (US)

(73) Assignee: integrated Nano-Technologies, Inc., Henrietta, NY (US)

(21) Appl. No.: 16/408,970

(22) Filed: May 10, 2019

Related U.S. Application Data
(63) Continuation-in-part of application No. 16/345,175, filed on Apr. 25, 2019.
(60) Provisional application No. 62/669,575, filed on May 10, 2018.
(60) Provisional application No. 62/413,144, filed on Oct. 26, 2016.

(51) Int. Cl. C12Q 1/6816; G01N 15/14; C12N 15/10; G01N 21/05; C12Q 1/6834

(52) U.S. Cl. CPC C12Q 1/6816; G01N 15/1475; C12N 15/101; B82Y 5/00; G01N 21/05; C12Q 1/6834; C12N 15/1013

(57) ABSTRACT

Systems and method for analyzing molecules in a sample. The system includes an imager, a flow cell, a magnet, and a processor. The flow cell includes a functionalized surface comprising a capture probe configured to bind molecules comprising a first RNA transcript. The magnet is positioned opposite the functionalized surface. The magnet is configured to direct the molecules comprising the first RNA transcript to the functionalized surface to bind to the capture probe. The light source configured to direct a light beam at the bound molecules comprising the first RNA transcript. The imager is configured to capture light from the bound molecules comprising the first RNA transcript. A processor configured to determine a quantity of the molecules in the sample comprising the first RNA transcript. The process is further configured to determine an expression level of the first RNA transcript in the sample based on the quantity of the molecules.

### CROSS-REFERENCE TO RELATED APPLICATIONS

This application claims the benefit of and priority to U.S. Provisional Patent Application Ser. No. 62/669,575, filed May 10, 2018, and entitled “ALZHEIMER′S SCREENING TECHNIQUE AND DEVICE,” and U.S. patent application Ser. No. 16/345,175, filed Apr. 25, 2019, which is a national stage entry under Section 371 of International Patent Application Serial No. PCT/US2017/058559, filed Oct. 26, 2017, which claims priority to U.S. Provisional Patent Application Ser. No. 62/413,144, filed Oct. 26, 2016 and entitled “AUTOMATED NUCLEIC ACID DETECTION AND QUANTITATION WITH OPTICAL SENSING,” the entirety of each which is incorporated herein by reference.

### BACKGROUND

The subject matter disclosed herein relates to detecting target molecules, such as nucleic acid molecules and, more particularly, to systems for optical sensing of the target molecules.

Various methods have developed for analyzing biological samples and detecting the presence of target molecules, such as nucleic acid molecules. These methods can be used, for example, in detecting pathogens in samples.

Typically, detection methods use disruption techniques, such as Polymerase Chain Reaction (PCR) to extract and replicate nucleic acid molecules from a sample. PCR is a technique that allows for replicating and amplifying trace amounts of DNA fragments into quantities that are sufficient for analysis. As such, PCR can be used in a variety of applications, such as DNA sequencing and detecting DNA fragments in samples.

An electronic sensor for detection of specific target nucleic acid molecules can include capture probes immobilized on a sensor surface between a set of paired electrodes. An example of a system and method for detecting target nucleic acid molecules is described in U.S. Pat. No. 7,645,574, the entirety of which is herein incorporated by reference. Following PCR, amplified products or amplicons derived from targeted pathogen sequences are captured by the probes. Nano-gold clusters, functionalized with a complementary sequence, are used for localized hybridization to the amplicons. Subsequently, using a short treatment with a gold developer reagent, the nano-gold clusters serve as catalytic nucleation sites for metallization, which cascades into the development of a fully conductive film. The presence of the gold film shorts the gap between the electrodes and is measured by a drop in resistance, allowing the presence of the captured amplification products to be measured. However, such sensors can be insensitive to small quantities of target molecules, resulting in false negative results or a failure to detect the target molecules.

### SUMMARY

In one embodiment, a system for analyzing molecules in a sample is presented. The system includes an imager, a flow cell, a magnet, and a processor. The flow cell includes a functionalized surface comprising a capture probe configured to bind molecules comprising a first RNA transcript. The magnet is positioned opposite the functionalized surface. The magnet is configured to direct the molecules comprising the first RNA transcript to the functionalized surface to bind to the capture probe. The light source configured to direct a light beam at the bound molecules comprising the first RNA transcript. The imager is configured to capture light from the bound molecules comprising the first RNA transcript. A processor configured to determine a quantity of the molecules in the sample comprising the first RNA transcript. The process is further configured to determine an expression level of the first RNA transcript in the sample based on the quantity of the molecules.

In another embodiment, a method for analyzing molecules in a sample is presented. The method uses an imager, a flow cell including a functionalized surface having a capture probe coupled to the functionalized surface, a magnet, and a light source. The method includes the following steps. Binding molecules comprising a first RNA transcript to magnetic particles. Directing the molecules comprising the first RNA transcript to the functionalized surface via the magnet. Binding the molecules comprising the first RNA transcript to the capture probe. Directing a light beam from the light source at the bound molecules comprising the first RNA transcript. Capturing light from the bound molecules comprising the first RNA transcript. Determining a quantity of the molecules in the sample comprising the first RNA transcript. Determining an expression level of the first RNA transcript in the sample based on the quantity of the molecules.

In another embodiment, a method for analyzing molecules in a sample is presented. The method uses an imager, a magnet, a light source, and a flow cell that includes a functionalized surface having a plurality of capture probes. Each of the plurality of capture probes is configured to bind molecules in the sample comprising one of the plurality of RNA transcripts. The method includes the following steps. Binding molecules in the sample to a magnetic particle. Directing the molecules to the functionalized surface using the magnet. Binding each specific molecule of the molecules to one of the plurality of capture probes configured to bind the RNA transcript of the specific molecule. Directing a light beam from the light source at bound molecules bound on each of the plurality of capture probes. Capturing light from the bound molecules. Determining a quantity of the bound molecules bound on each of the plurality of capture probes based on the captured light. Determining a plurality of expression levels corresponding to the plurality of RNA transcripts based on the quantity of the bound molecules bound on each of the plurality of capture probes configured to bind each of the plurality of RNA transcript.

The above embodiments are exemplary only. Other embodiments are within the scope of the disclosed subject matter.

### BRIEF DESCRIPTION OF THE DRAWINGS

So that the manner in which the features of the invention can be understood, a detailed description of the invention may be had by reference to certain embodiment, some of which are illustrated in the accompanying drawings. It is to be noted, however, that the drawings illustrate only certain embodiments of this invention and are therefore not to be considered limiting of its scope, for the scope of the disclosed subject matter encompasses other embodiments as well. The drawings are not necessarily to scale, emphasis generally being placed upon illustrating the features of certain embodiments of the invention. In the drawings, like numerals are used to indicate like parts throughout the various views.

FIG. 1 is a perspective view of a portable diagnostic assay system operative to accept one of a plurality of disposable cartridges configured to test fluid samples of collected blood/food/biological samples;

FIG. 2 is an exploded perspective view of one of the disposable cartridges configured to test a blood/food/biological sample;

FIG. 3 is a top view of the one of the disposable cartridges illustrating a variety of assay chambers including a central assay chamber, one of which contains an assay chemical suitable to breakdown the fluid sample to detect a particular attribute of the tested fluid sample;

FIG. 4 is a bottom view of the disposable cartridge shown in FIG. 3 illustrating a variety of channels operative to move at least a portion of the fluid sample from one chamber to another for the purpose of performing multiple operations on the fluid sample.

FIG. 5 is a diagram of an embodiment of a sensor system having a functionalized surface;

FIG. 6 is a flowchart illustrating an embodiment of a method of detecting a target molecule;

FIG. 7 is a diagram of the sensor system of FIG. 5 with target molecules bound to magnetic particles;

FIG. 8A is cross-sectional illustration of an embodiment of a magnetic particle;

FIG. 8B is a cross-sectional illustration of another embodiment of a magnetic particle;

FIG. 8C is an illustration of an embodiment of a magnetic particle bound with nanoparticles;

FIG. 8D is an illustration of another embodiment of a magnetic particle bound with nanoparticles;

FIG. 8E is an illustration of an embodiment of a target molecule bound with a magnetic particle and a nanoparticle;

FIG. 8F is an illustration of another embodiment of a target molecule bound with a magnetic particle and nanoparticles;

FIG. 8G is an illustration of yet another embodiment of a target molecule bound with a nanoparticle and magnetic particles;

FIG. 9 is a diagram of the sensor system of FIGS. 5 and 7 with the target molecules bound to the functionalized surface;

FIG. 10 is a diagram of the sensor system of FIGS. 5 and 7-8 with functionalized nanoparticles bound to the target molecules;

FIG. 11 is a diagram of the sensor system of FIGS. 5, 7-8, and 10 with a light source directed at the functionalized nanoparticles;

FIG. 12A is an embodiment of scattering signatures of 50 nm monodispersed nanoparticles under dark field microscopy;

FIG. 12B is an embodiment of scattering signatures of 100 nm monodispersed nanoparticles under dark field microscopy;

FIG. 13 is a comparison of scattering signatures of developed nanoparticles versus undeveloped nanoparticles under dark field microscopy;

FIG. 14 is an illustration of an embodiment of an optical sensor system;

FIG. 15A is an enlarged partial illustration of the optical sensor system of FIG. 14 with the magnet retracted;

FIG. 15B is an enlarged partial illustration of the optical sensor system of FIG. 14 with the magnet extended;

FIG. 16 is a side view illustration of an embodiment of an optical instrument incorporating the optical sensor system of FIG. 14;

FIG. 17A is a diagram of another embodiment of a sensor system having a functionalized surface;

FIG. 17B is a diagram of the sensor system of FIG. 17A with target molecules bound to the functionalized surface;

FIG. 17C is a diagram of the sensor system of FIGS. 17A-17B having nanoparticles bound to the target molecules;

FIG. 18 depicts a method for analyzing a plurality of RNA transcripts in a sample;

FIG. 19 depicts a method for determining a risk level of a neurodegenerative disease by analyzing a plurality of RNA transcripts of a sample; and

FIG. 20 depicts another working example.

Corresponding reference characters indicate corresponding parts throughout several views. The examples set out herein illustrate several embodiments, but should not be construed as limiting in scope in any manner.

### DETAILED DESCRIPTION

A disposable cartridge is described for use in a portable/automated assay system such as that described in commonly-owned, U.S. patent application Ser. No. 15/157,584 filed May 18, 2016 entitled “Method and System for Sample Preparation” which is hereby included by reference in its entirety. While the principal utility for the disposable cartridge includes DNA testing, the disposable cartridge may be used to detect any of a variety of diseases which may be found in either a blood, food or biological detecting hepatitis, autoimmune deficiency syndrome (AIDS/HIV), diabetes, leukemia, graves, lupus, multiple myeloma, etc., just naming a small fraction of the various blood borne diseases that the portable/automated assay system may be configured to detect. Food diagnostic cartridges may be used to detect salmonella, e-coli, staphylococcus aureus or dysentery. Diagnostic cartridges may also be used to test samples from insects and specimen. For example, blood diagnostic cartridges may be dedicated cartridges useful for animals to detect diseases such as malaria, encephalitis and the west Nile virus, to name but a few.

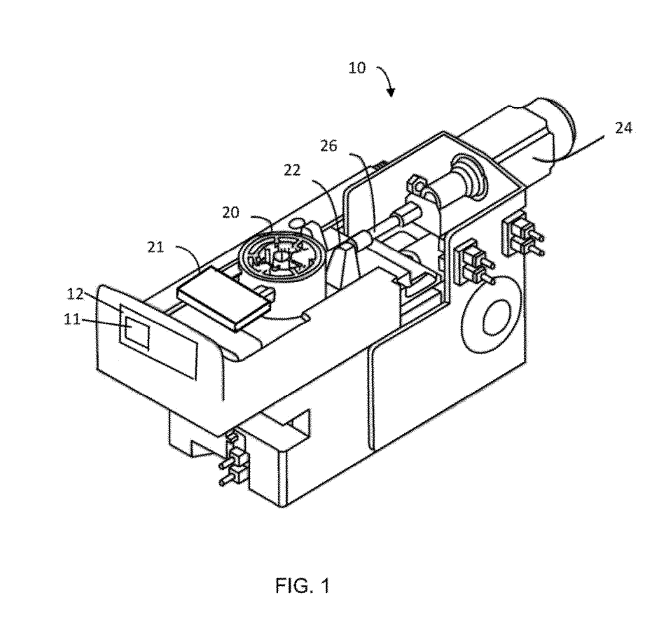

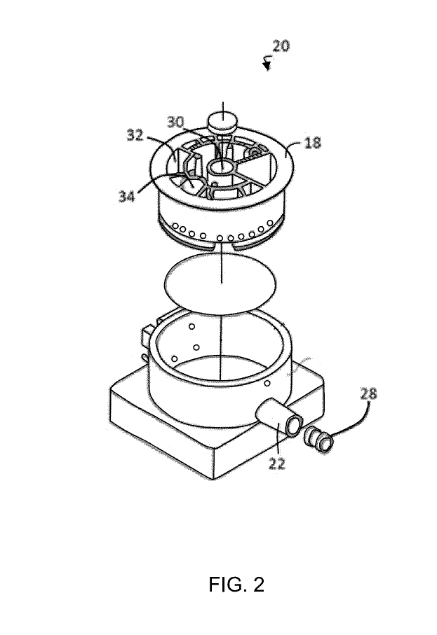

More specifically, and referring to FIGS. 1 and 2, a portable assay system 10 receives any one of a variety of disposable assay cartridges 20, each selectively configured for detecting a particular attribute of a fluid sample, each attribute potentially providing a marker for a blood, food or biological (animal borne) disease. The portable assay system 10 includes one or more linear and rotary actuators operative to move fluids into, and out of, various compartments or chambers of the disposable assay cartridge 20 for the purpose of identifying or detecting a fluid attribute. More specifically, the cartridge 20 includes a flow cell 21 extending horizontally therefrom. A rotary actuator (not shown) of the portable assay system 10 aligns one of a variety of ports 18P, disposed about a cylindrical rotor 18, with a syringe barrel 22B of a stationary cartridge body 22. The linear actuator 24 displaces a plunger shaft 26 so as to develop pressure i.e., positive or negative (vacuum) in the syringe barrel 22. That is, the plunger shaft 26 displaces an elastomer plunger 28 within the syringe 22 to move and or admix fluids contained in one or more of the chambers 30, 32. In addition, system 10 includes one or more processors 11 housed within a control board 12 of the body of the system 10 for receiving signals from the various components of the system 10.

The disposable cartridge 20 provides an automated process for preparing the fluid sample for analysis and/or performing the fluid sample analysis. The sample preparation process allows for disruption of cells, sizing of DNA and RNA, and concentration/clean-up of the material for analysis. More specifically, the sample preparation process of the instant disclosure prepares fragments of DNA and RNA in a size range of between about 100 and 10,000 base pairs. The chambers can be used to deliver the reagents necessary for end-repair and kinase treatment. Enzymes may be stored dry and rehydrated in the disposable cartridge 20, or added to the disposable cartridge 20, just prior to use. The implementation of a rotary actuator allows for a single plunger 26, 28 to draw and dispense fluid samples without the need for a complex system of valves to open and close at various times. This greatly reduces potential for leaks and failure of the device compared to conventional systems. Finally, it will also be appreciated that the system greatly diminishes the potential for human error.

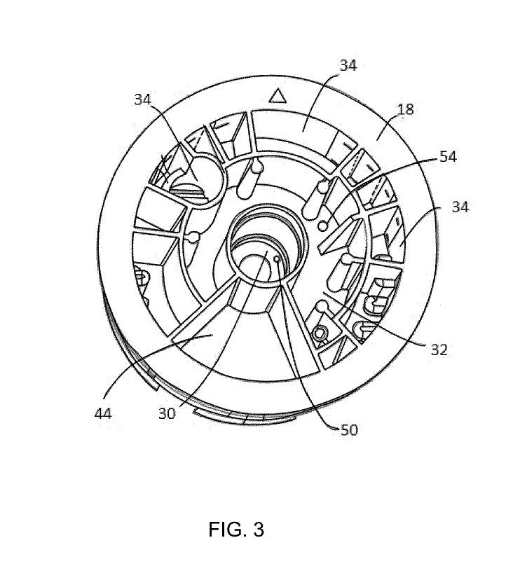

In FIGS. 3 and 4, the cylindrical rotor 18 includes a central chamber 30 and a plurality of assay chambers 32, 34 surrounded, and separated by, one or more radial or circumferential walls. In the described embodiment, the central chamber 30 receives the fluid sample while the surrounding chambers 32, 34 contain a premeasured assay chemical or reagent for the purpose of detecting an attribute of the fluid sample. The chemical or reagents may be initially dry and rehydrated immediately prior to conducting a test. Some of the chambers 32, 34 may be open to allow the introduction of an assay chemical while an assay procedure is underway or in-process. The chambers 30, 32, 34 are disposed in fluid communication, i.e., from one of the ports 18P to one of the chambers 30, 32, 34, by channels 40, 42 molded along a bottom panel 44, i.e., along underside surface of the rotor 18. For example, a first port 18P, corresponding to aperture 42, may be in fluid communication with the central chamber 30, via aperture 50.

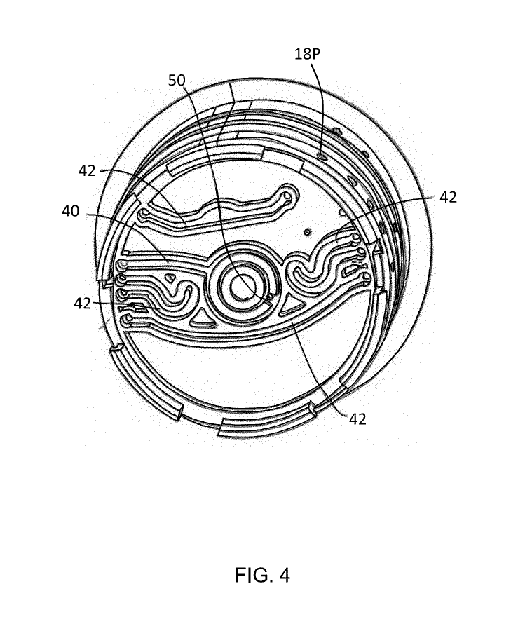

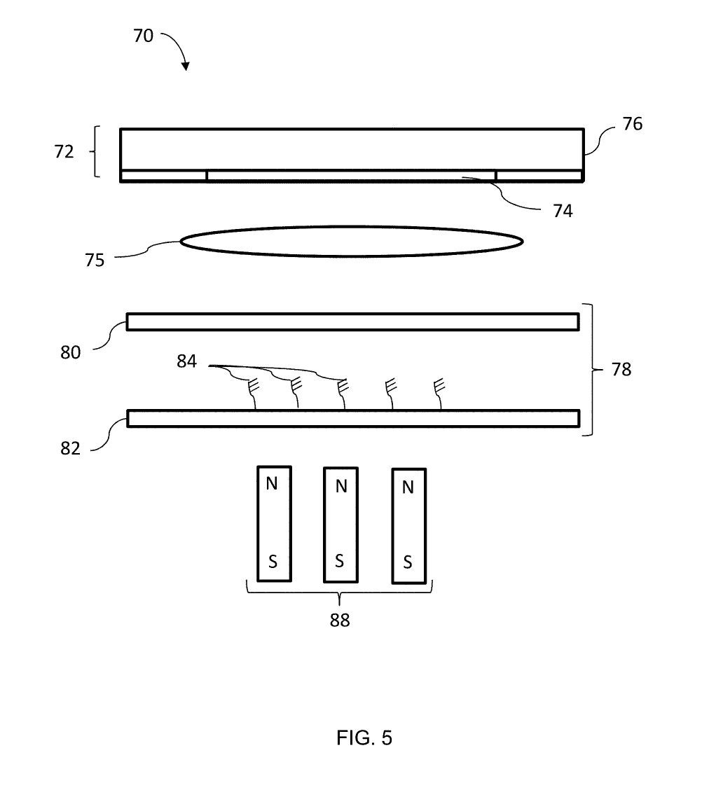

FIG. 5 illustrates an embodiment of a sensor system 70. The sensor system 70 includes an imager 72 configured to capture still images, video, or a combination thereof For example, the imager 72 can be configured to capture high resolution still images. In the illustrated embodiment, the imager 72 includes a pixel array 74 and array circuitry 76. The pixel array 74 can include any suitable number of pixels. For example, the pixel array 74 can be a high density array including at least six (6) megapixels. In a further example, the camera can have a large field of view. The pixel array 74 is a light sensitive pixel array, such as an active array, passive array, planar Fourier capture array, angle sensitive array, photodiode array, a charge coupled device, a complementary metal-oxide semiconductor (CMOS), or a charge injection device.

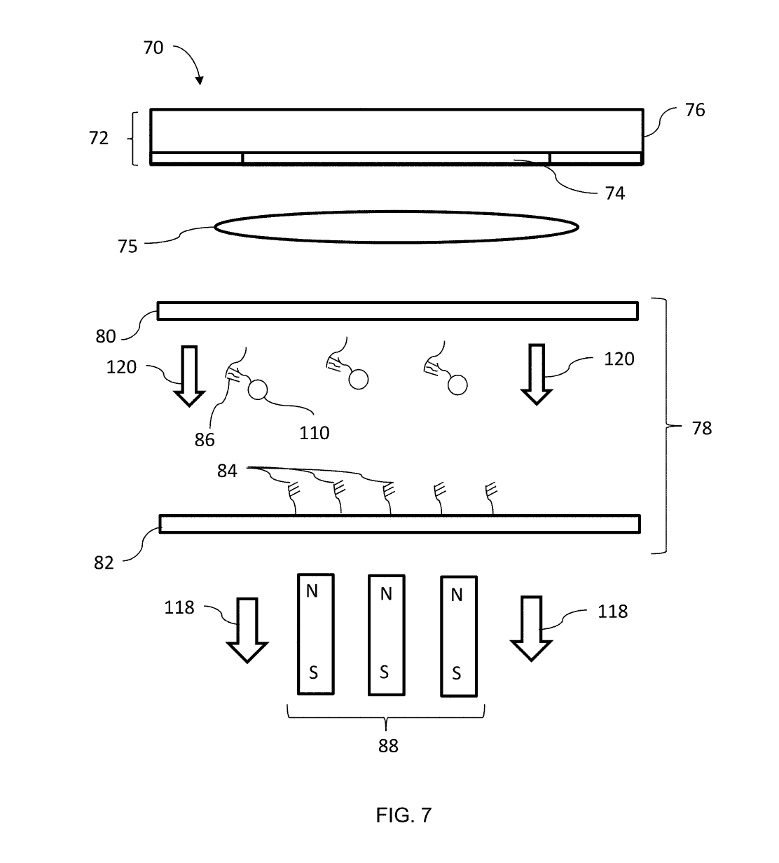

The sensor system 70 also includes a flow cell 78. The flow cell 78 can be formed of any suitable material, such as a polypropylene or polystyrene polymer or glass, among others. In an embodiment, the flow cell is formed by injection molding. The flow cell 78 includes a transparent or optically clear surface 80 and a transparent functionalized surface 82. The functionalized surface 82 includes a plurality of capture probes 84 in the form of a functionalized oxide surface allowing attachment and immobilization of capture probe molecules 84 on the surface 82. The capture probes 84 are designed to capture or bind target molecules 86 (FIG. 7) by interaction between complementary sequences. The target molecules 86 can be collected from a biological sample. The biological sample could be any suitable type of materials, such as blood, mucous, and skin, among others. For example, the target molecules 86 can be protein ligands or DNA segments.

An objective or lens 75 can optionally be positioned between the imager 72 and the flow cell 78. A magnet 88 can be positioned opposite the functionalized surface 82. The magnet 88 can be a single magnet or an array of magnets.

In one embodiment, the functionalized surface 82 includes an array of many different capture probes 84, and each capture probe 84 is configured to capture molecules having different RNA transcripts. Thus, the array of probes 84 can be deployed to capture, say, between 10-20 different RNA transcripts. In another example, multiple probes 84 may be configured to capture molecules having the same RNA transcripts, so that there is redundancy to allow for error checking of the results. Thus, an array of capture probes 84 can be designed to capture many different RNA transcripts to analyze the relative proportion of molecules in the sample that are expressing those RNA transcripts. In another example, one or more of the array of probes 84 can be configured to capture so-called housekeeping genes, which can serve as a baseline by which to normalize the number of other molecules found on other genes. For instance, if 400 molecules of the housekeeping gene are captured, then the number of a different captured molecule can be compared to 400 to give an absolute sense of how many of those molecules are captured as a percentage compared to the housekeeping genes, thus establishing an absolute scale.

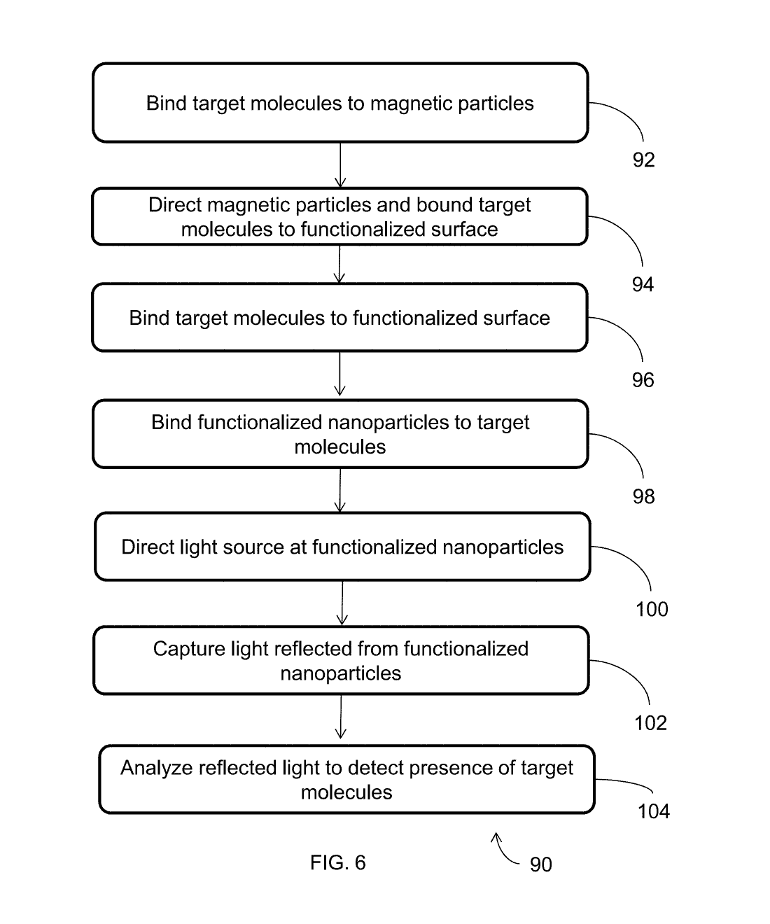

FIG. 6 illustrates an embodiment of a method 90 for detection of a target molecule. The method 90 can be employed by a sensor system, such as the sensor system 70. At block 92, target molecules 86 are bound to magnetic particles 110, as illustrated in FIG. 7. In an embodiment, the target molecules 86 are bound to the magnetic particles 110 before being introduced to the flow cell 78. In another embodiment, the target molecules 86 and magnetic particles 110 are introduced to the flow cell 78 in an unbound state and the target molecules 86 bind to the magnetic particles 110 within the flow cell 78.

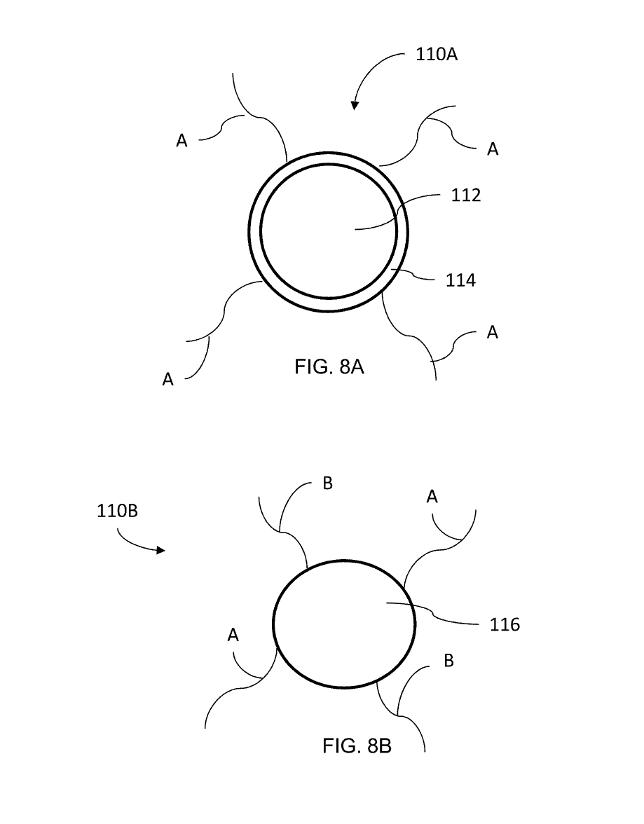

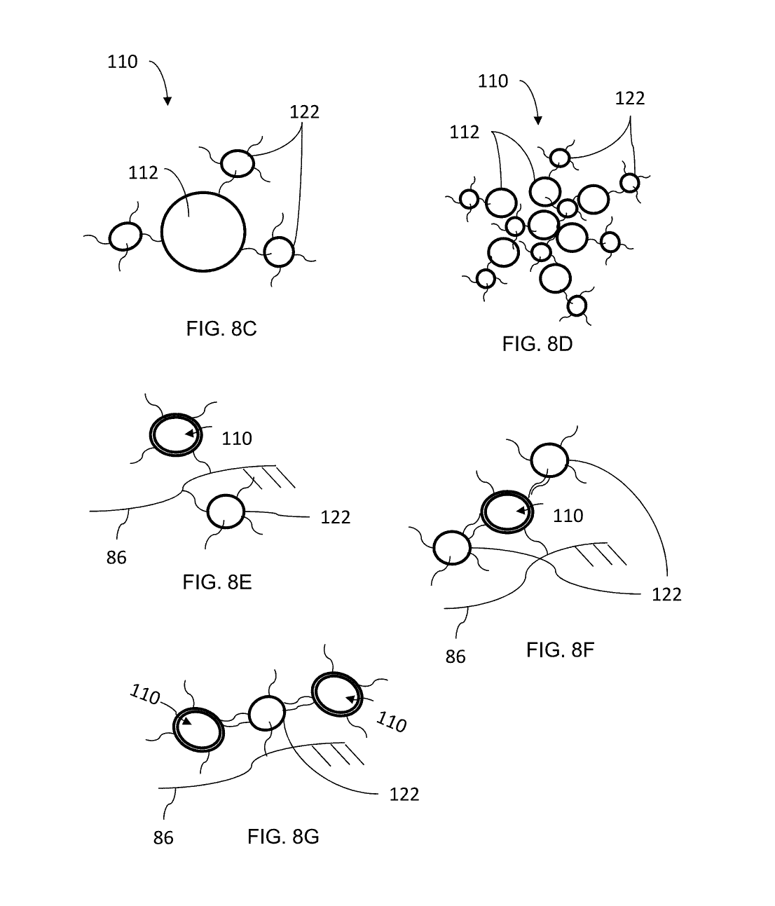

FIGS. 8A-8B illustrate two embodiments of magnetic particles 110. As illustrated in FIG. 8A, in one embodiment the magnetic particle 110A is a composite particle that has a magnetic core 112, formed of a magnetic material such as iron, and a nanoparticle coating 114. For example, the coating 114 can be a gold coating. The coating 114 can be configured to act as a nucleation site for further nanoparticle development. The magnetic particle 110A includes at least one binding site for a ligand A for binding to the target molecules 86. A chemical reactive group such as a thiol, amine, or aldehyde, can mediate or facilitate ligand binding.

As illustrated in FIG. 8B, in another embodiment, the magnetic particle 110B can have a magnetic body 116 formed of a magnetic material, such as iron. The magnetic particle 110B includes at least one binding site or ligand A for binding to a target molecule. In the illustrated embodiment, the magnetic particle 110B further includes at least one binding site or ligand B for binding a magnetic nanoparticle. It is to be understood that the magnetic particle can include any suitable combination of binding sites A, B. For example, the magnetic particle can include both types of binding sites A, B or the magnetic particle can include only target molecule binding sites A.

As illustrated in FIG. 8C, rather than a nanoparticle coating over a magnetic core, the magnetic particle 110 can include a magnetic body 112 and a plurality of nanoparticles 122 bound to the magnetic body 112. Alternatively, as illustrated by FIG. 8D, the magnetic particle 110 can be an alloy, such as a heterogeneous alloy, including a plurality of magnetic bodies 112 bound with a plurality of nanoparticles 122.

As illustrated in FIGS. 8E-8G, the target molecule 86, magnetic particle 110, and nanoparticle 122 can be bound in a variety of arrangements. As illustrated in FIG. 8E, the magnetic particle 110 and nanoparticle 122 can each be bound directly to the target molecule 86. Alternatively and as illustrated in FIG. 8F, the magnetic particle 110 can be bound directly to the target molecule 86 and one or more nanoparticles 122 can be bound to the magnetic particle 110. Alternatively and as illustrated in FIG. 8G, a nanoparticle 122 can be bound directly to the target molecule 86 and one or more magnetic particles 110 can be bound to the nanoparticle 122.

Returning to FIG. 6, at block 94, the bound magnetic particles 110 and target molecules 86 are directed or moved to the functionalized surface 82. Referring to FIG. 7, the magnet 88 is coupled to an actuator (not shown) configured to move the magnet 88 toward (retracted) and away from (extended) the functionalized surface 82. As the magnet 88 is moved away 118 from the functionalized surface 82, the magnetic particles 110, attracted to the magnet 88, move 120 toward the functionalized surface 82. As the target molecules 86 are bound to the magnetic particles 110, the target molecules 86 are directed or drawn by the magnetic particles 110 toward the functionalized surface 82.

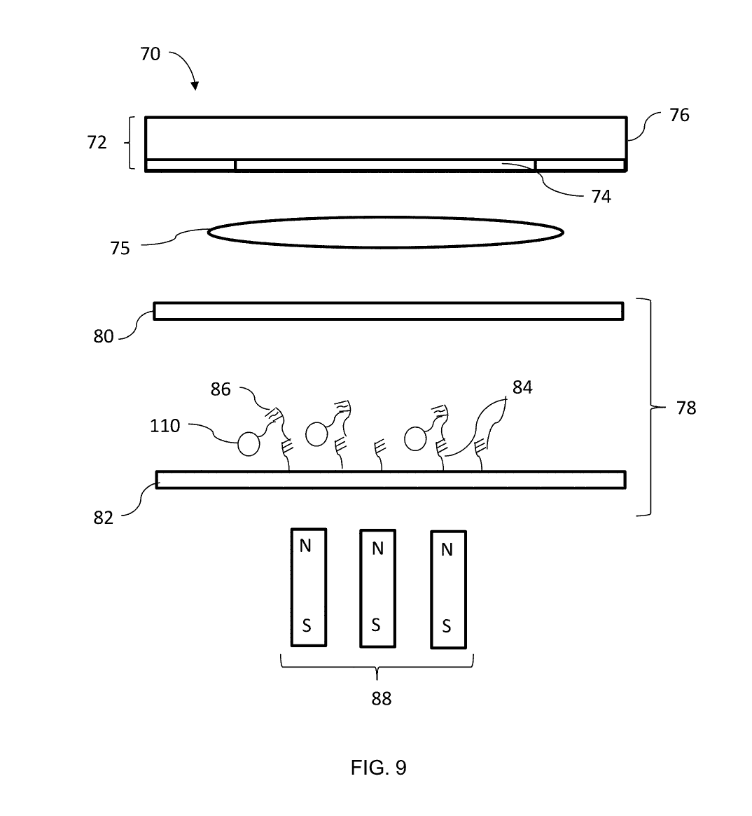

Returning to FIG. 6, at block 96, the target molecules 86 are bound to the capture probes 84 of the functionalized surface 82 as illustrated in FIG. 9. In the illustrated embodiment, the magnetic particles 110 remain bound to the bound target molecules 86. Alternatively, the target molecules 86 can be denatured to unbind the magnetic particles 110 from the target molecules 86 when the target molecules 86 reach the functionalized surface 82, following which the target molecules 86 can bind to the functionalized surface 82.

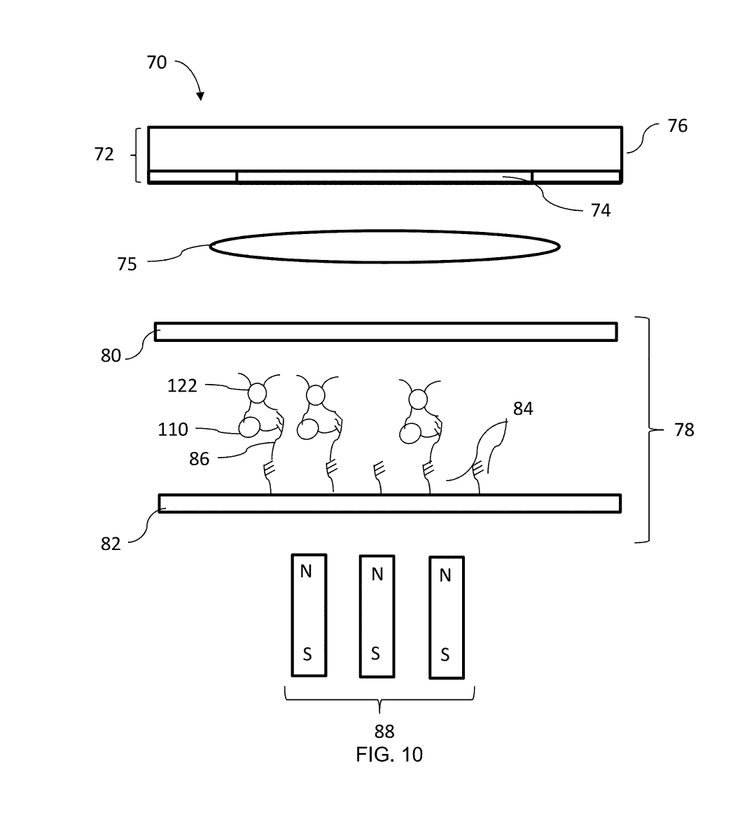

Returning to FIG. 6, at block 98, functionalized nanoparticles 122 are introduced to the flow cell 78 and are bound to the target molecules 86, as illustrated in FIG. 10. In an embodiment, the functionalized nanoparticles 122 are bound directly to the target molecules 86. Alternatively, the functionalized nanoparticles 122 are bound to the magnetic particles 110 bound to each target molecules 86. In an embodiment, a plurality of functionalized nanoparticles 122 are bound to each target molecule 86. Any suitable method of hybridizing or binding the nanoparticles 122 to the target molecules 86 can be used. In an embodiment, the functionalized nanoparticle 122 is a gold particle. In another embodiment, the functionalized nanoparticle 122 is a catalytic nanoparticle, such as a gold catalyst reagent. In an embodiment, the nanoparticles 122 are in the form of catalyst clusters.

In the illustrated embodiment, the nanoparticles 122 are bound to the target molecules 86 after the target molecules 86 are bound to the functionalized surface 82. In an alternative embodiment, the nanoparticles 122 can be bound to the target molecules 86 or magnetic particles 110 prior to binding of the target molecules 86 to the functionalized surface 82.

Following binding of the nanoparticles 122 to the target molecules 86, an optional metallization step can be performed to metallize the nanoparticles 122 and develop or form enlarged nanoparticles or even a film. The developed nanoparticles can improve detection of the target molecules. In this metallization step, the nanoparticles 122 serve as nucleation sites for development of enlarged nanoparticles 124.

Returning to FIG. 6, at block 100, a light source 126 directs a light beam 128 at the target molecules 86 and functionalized nanoparticles 122, 124 in the flow cell 78. The light beam 128 is aimed so that light is directed solely at the target molecules 86 and nanoparticles 122, 124 and no light 128 from the light source 126 is captured by the imager 72. In an embodiment, the flow cell 78 is configured to prevent diffusion of the light beam 128 toward the imager 72.

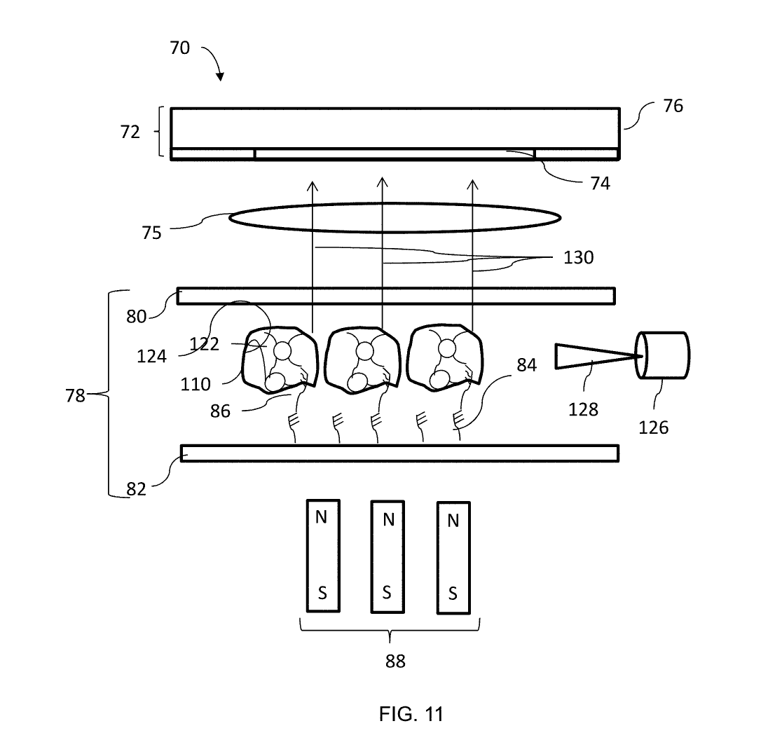

Referring to FIG. 6, at block 102, light 130 (FIG. 11) from the nanoparticles 122, 124, target molecules 86, magnetic particles 110, or a combination thereof, is captured by the imager 72. In an embodiment, the light 130 can be reflected or emitted from the particles 86, 110, 122, 124, or a combination thereof. At block 104, the light 130 captured by the imager 72 is analyzed to detect the number of target molecules 86 present. For example, the captured light 130 can be analyzed using dark field microscopy. In this embodiment, the spots of detected light are counted and quantified to determine the number of target molecules 86 present. Counting and quantifying is accomplished using the one or more processors 11 (see FIG. 1).

In one embodiment, a single imager 72 and light beam 128 may be used as described above to sequentially examine each of the capture probes 84. Such sequential analysis may be achieved by providing, for example, for translation of the imager 72 and light beam 128 across the flow cell to allow each capture probe 84 to be examined one at a time. In another embodiment, adjustable mirrors can be used to direct the light from the light beam 128 to one of the capture probes 84 and the imager 72 one at a time by adjusting the mirrors. In a further embodiment, an imager with a larger imaging capability can be fixed with the light beam 128 moving to each capture probe 84 one at a time. In another embodiment, multiple imagers 72 can be used, with each one dedicated to one or several capture probes 84.

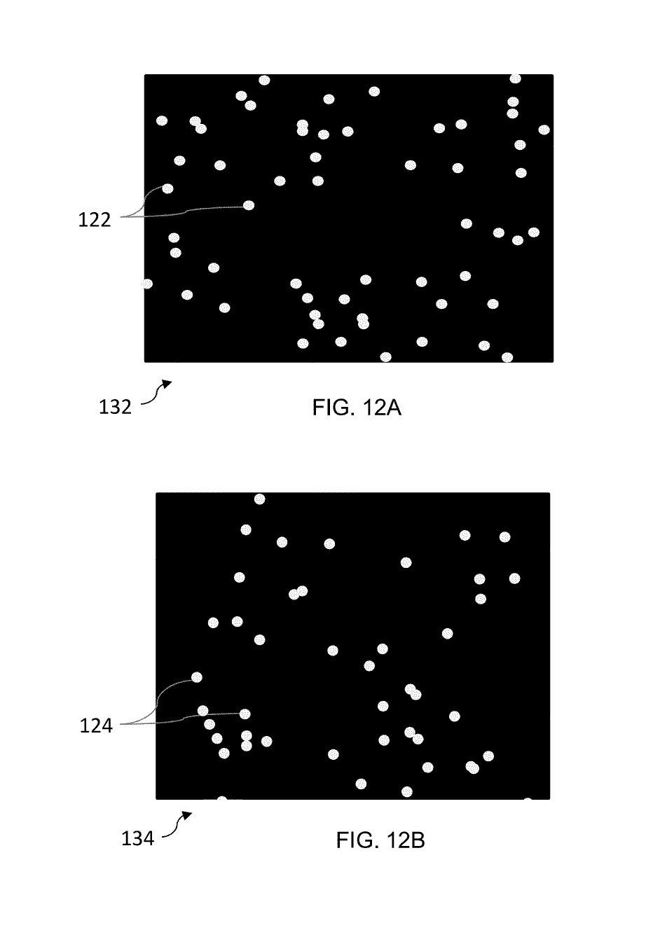

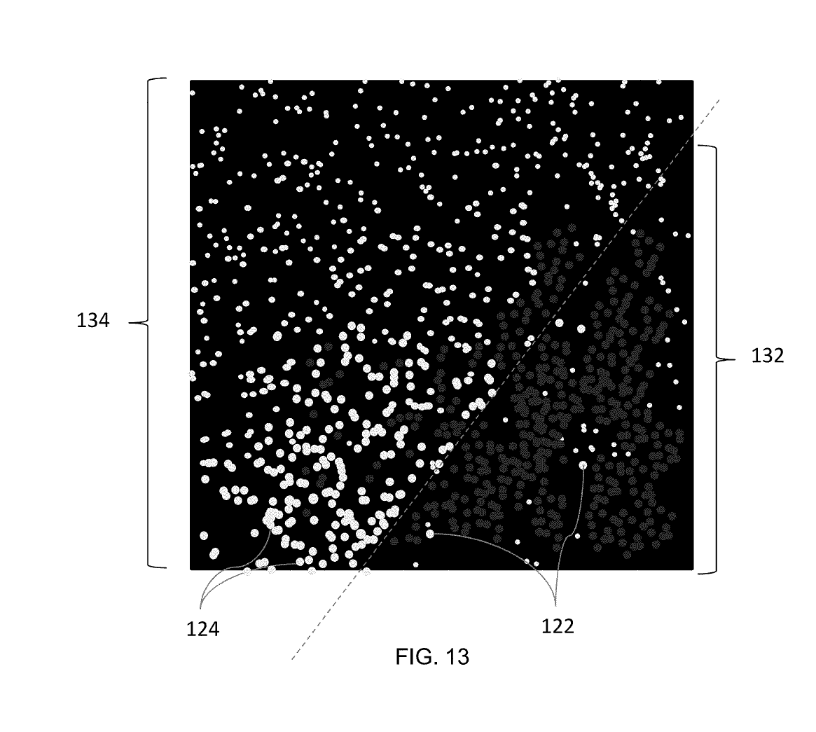

FIGS. 12A-13 illustrate embodiments of light or scattering signatures or captured images of gold nanoparticles 122 captured under dark field microscopy. FIG. 12A is a scattering signature of 50 nm gold nanoparticles and FIG. 12B is a scattering signature of 100 nm gold nanoparticles. FIG. 13 is an image comparing the scattering signature 132 of undeveloped (20 nm) nanoparticles 122 with the scattering signature 134 of developed (100 nm) nanoparticles 124. In an embodiment, a series of dark field images can be captured. In this embodiment, a first image can be captured prior to development of the nanoparticles 122 and at least one additional image can be captured as the nanoparticles are developed. Alternatively, a first image can be captured prior to binding of the target molecules 86 and at least one additional image can be captured following binding of the nanoparticles 122. The captured images can be compared to removed background artifacts and improve analysis of the dark field images.

In an alternative embodiment, a dye particle (not shown) is coupled to the target molecules 86 for detection of the target molecules 86. In this embodiment, the light source 128 is tuned to the wavelength of the dye and regions covered by the dye will fluoresce. The fluoresce is detected by the imager 72.

In another alternative embodiment, to detect the presence of the target molecules 86, following binding of the target molecules 86 and nanoparticles 122, the functionalized surface is exposed to a radiation source (not shown). Upon exposure to the radiation source, the regions of nanoparticles preferentially absorb the radiation, causing localized heating. The localized heating is captured and registered by the imager 72 to detect the presence of the target molecules 86. Based on the amount of heating registered, a count of the number of target molecules present is established. The system may be calibrated by allowing a known number of target molecules to be heated, and measuring the temperature, for example.

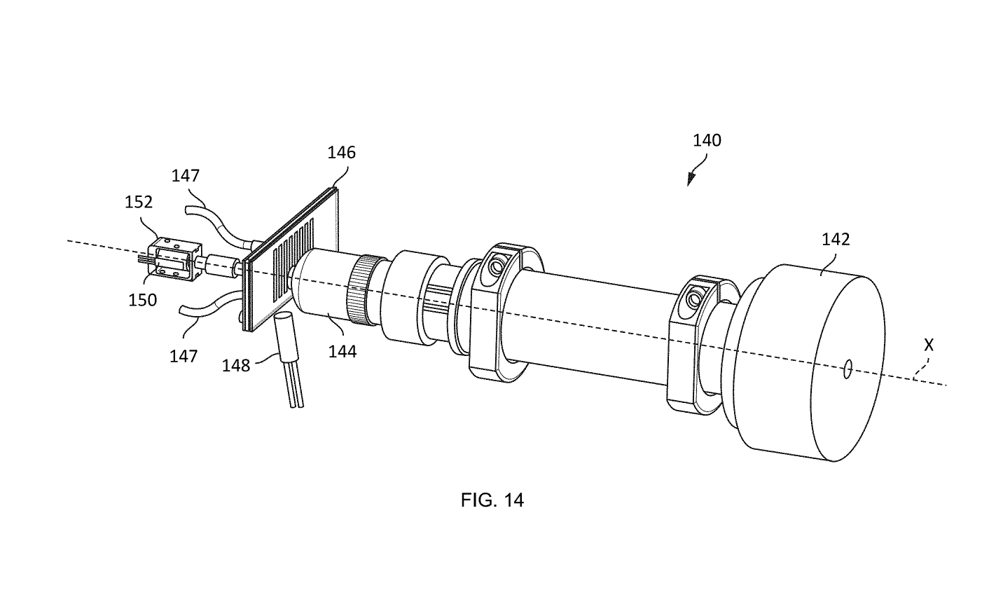

An example of an optical sensor system 140 is illustrated in FIG. 14. Similar to the optical sensor system 70 described above, the optical sensor system 140 includes an imager 142 and an objective or lens 144 coupled to the imager 142. In an embodiment, the imager 142 is a high resolution imager having a wide angle or large field of view. The objective 144 is directed toward the flow cell 146. As discussed above, the interior of the flow cell 146 includes a functionalized surface for binding target molecules. One or more feeder lines 147 can be coupled to the flow cell 146 to facilitate the introduction of various particles to the flow cell 146.

A light source 148 is directed at the flow cell 146. The light source 148 can be any suitable light source. For example, the light source 148 can provide light at a predetermined frequency. For example, the light source 148 can be a white light. The light source 148 is directed or aimed solely at the flow cell 146. In the illustrated embodiment, the light source 148 is directed orthogonally to the axis X on which the objective is positioned. The flow cell 146 is configured to channel the light from the light source 148 toward the particles within the flow cell 146, rather than toward the imager 142 and to prevent light diffusion from the light source 148 to the imager 142.

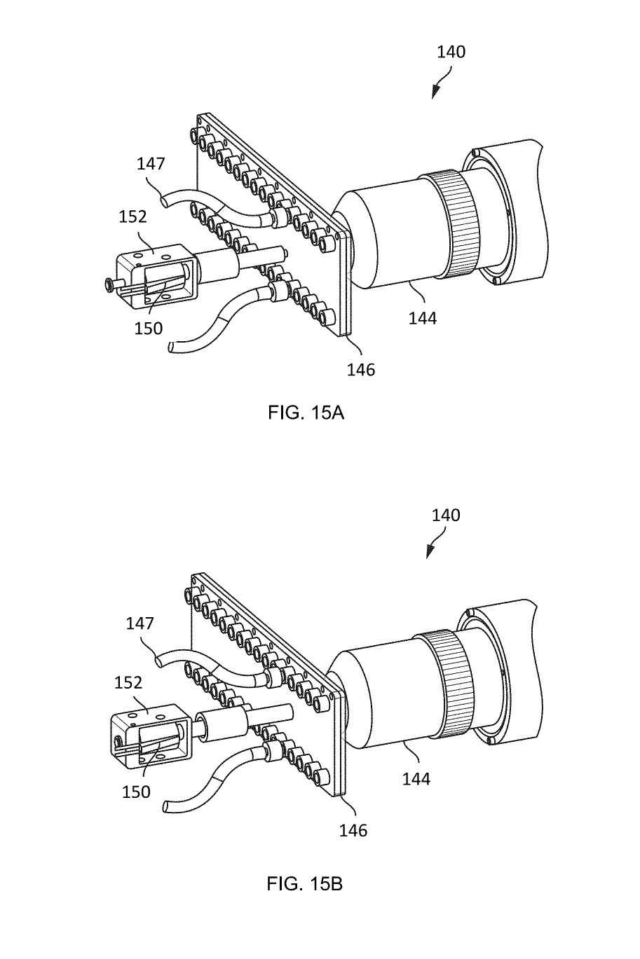

A magnet 150 is positioned opposite the flow cell 146 from the objective 144. An actuator 152, such as a solenoid, is coupled to the magnet 150 and is configured to move the magnet. As illustrated in FIGS. 15A-15B, in an embodiment, the actuator 152 is configured to retract or move the magnet 150 toward (FIG. 15A) the flow cell 146 and to extend or move the magnet 150 away (FIG. 15B) from the flow cell 146.

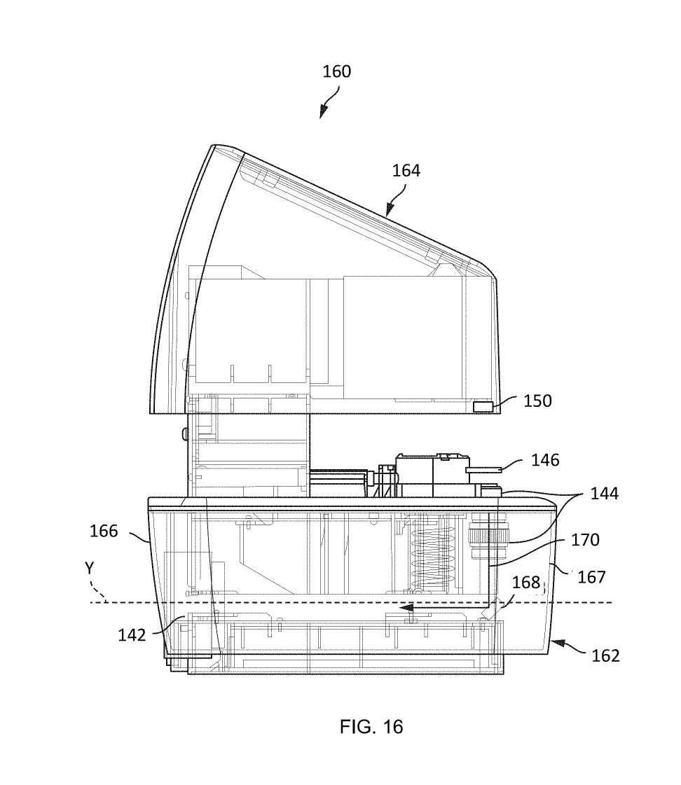

FIG. 16 illustrates an embodiment of an analysis system 160 including an optical system, such as the optical sensor systems 70, 140. In this embodiment, the analysis system 160 includes a base 162 and a head 164. The imager 142 and objective 144 are positioned in the base 162. The flow cell 146 is positioned on the top surface of the base 162, aligned with the objective 144. The magnet 150 is positioned in the head 164 and is configured to extend to and engage with the flow cell 146.

In the illustrated embodiment, in order to minimize the footprint of the analysis system 160, the imager 142 and objective 144 are not aligned along an axis, as illustrated in FIG. 14. Rather, the imager 142 is aligned along an axis Y extending longitudinally through the base 162 between the side surfaces 166, 167 of the base 162. The objective 144 is positioned orthogonal to the axis Y and extends upward through the base 162. A mirror 168 is positioned below the objective 144 and angled toward the imager 142 to create an optical path 170 between the objective 144 and the imager 142.

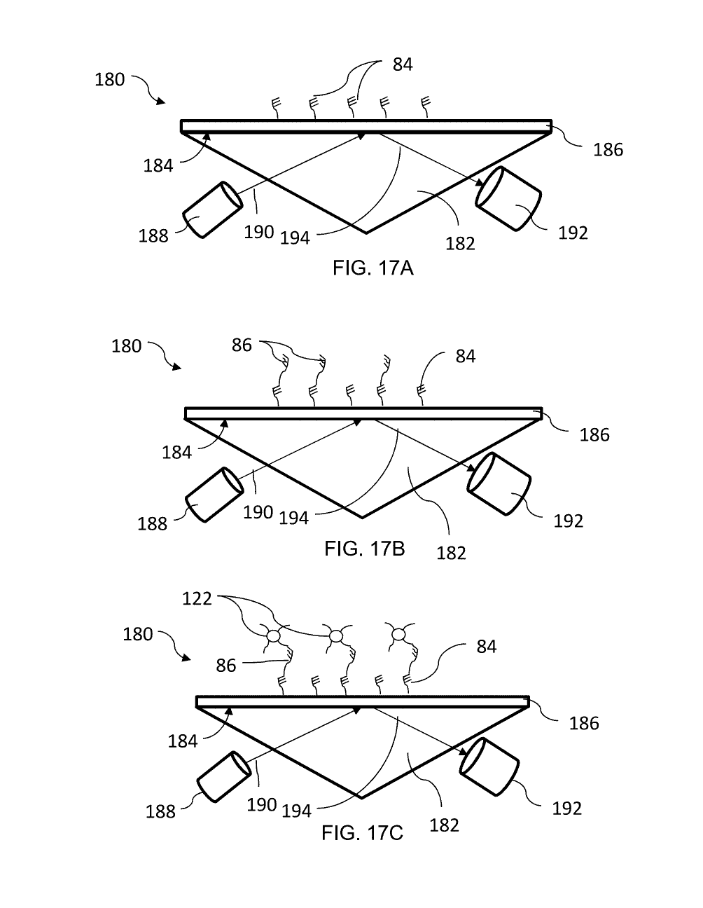

FIGS. 17A-17C illustrate an alternative embodiment of a sensor system 180. In this embodiment. The sensor system 180 includes a prism type substrate 182 having a Kretshmann configuration. The substrate 182 has a surface 184 coated with a metal film 186 suitable for surface plasmon resonance or Raman scattering. For example, the metal film can be gold, silver, copper, titanium, or chromium. The film 186 is functionalized with a bio-specific coating to include capture probes 84. A light source 188 directs a light beam 190 through the prism substrate 182 toward the film 186 and a detector 192 captures light 194 from the film 186, such as light reflected or emitted by the film 186.

In operation, a baseline measurement of the captured light is taken. In an embodiment, the baseline measurement of the captured light is used to calibrate the absorbance angle (FIG. 17A). In addition, the baseline measurement can be sued to identify contaminants or debris on the sensor prior to binding of the target molecules 86 or prior to development of increased nanoparticle size, as discussed below. Following the baseline measurement, a sample containing target molecules 86 is introduced to the sensor system 180 and the target molecules 86 bind to the capture probes 84 (FIG. 17B). In an embodiment, the target molecules 86 can be directed to the surface film 186 via magnetic particles as described above. Functionalized nanoparticles 122 are introduced to the system 180 and allowed to bind to the target molecules 86 (FIG. 17C). A plating bath can optionally be used to increase the size of the bound nanoparticles 122. To detect the presence of the target molecules 86, the beam 190 is directed toward the film 186 and the light 194 from the film 186, such as reflected or emitted, is captured. Any difference in reflectivity or intensity between the baseline measurement and the final measurement is observed in order to detect the presence of the target molecules 86. A quantitative count of target molecules 86 may be established by comparison with calibrated tests of the reflectivity or intensity for known number of target molecules. In an embodiment, the baseline measurement can be used to subtract particles identified as debris from the final measurement.

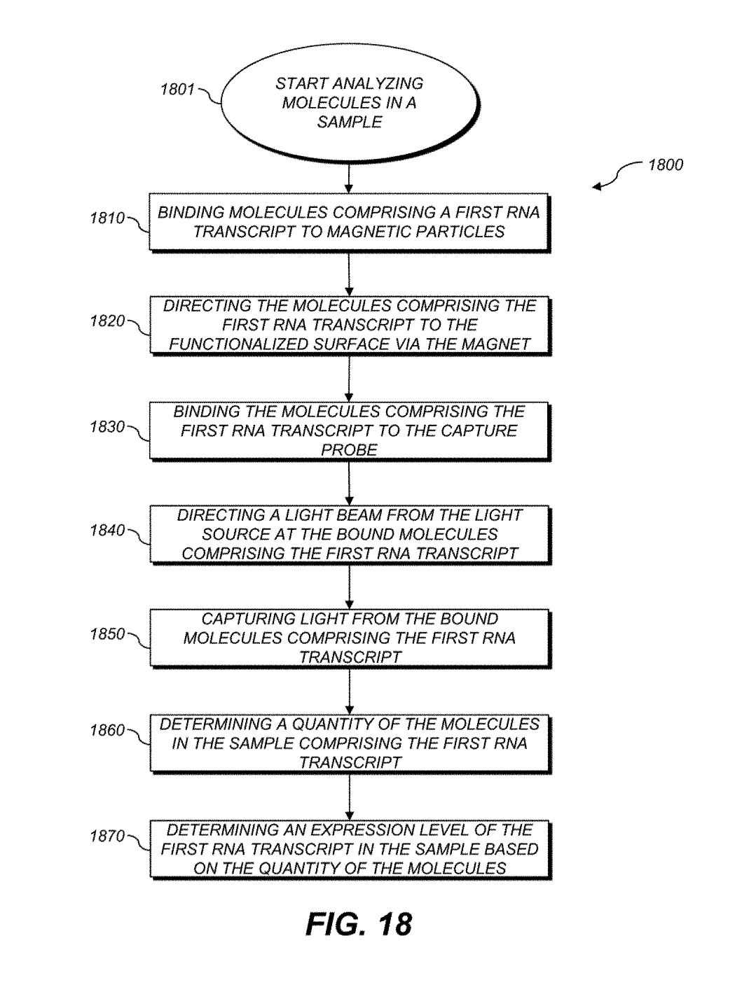

FIG. 18 depicts a method 1800 for analyzing a plurality of RNA transcripts in a sample.

By way of background, several technologies have made it possible to monitor the expression level of a large number of transcripts within a cell at any one time (see, e.g., Schena et al., 1995, Quantitative monitoring of gene expression patterns with a complementary DNA micro-array, Science 270:467-470; Lockhart et al., 1996, Expression monitoring by hybridization to high-density oligonucleotide arrays, Nature Biotechnology 14:1675-1680; Blanchard et al., 1996, Sequence to array: Probing the genome's secrets, Nature Biotechnology 14, 1649; 1996, U.S. Pat. No. 5,569,588, issued Oct. 29, 1996 to Ashby et al. entitled “Methods for Drug Screening”).

Applications of transcript array technology have involved identification of genes which are up regulated or down regulated in various diseased states. Additional uses for transcript arrays have included the analyses of members of signaling pathways, and the identification of targets for various drugs. Transcript arrays can be beneficial in monitoring the level of either disease states or effect of therapies.

RNA profiling is a process useful to monitor disease state or treatment efficacy by monitoring the expression levels of key RNA transcripts in a sample. Key RNA transcripts can be identified using techniques such as an Affymetrix array to screen all transcripts in a cell. By profiling multiple patients at different stages of disease, a set of key indicator transcripts can be identified and used on an array, such as that of the current invention which targets the key indicators. In addition, some housekeeping genes which do not vary due to the disease are also monitored to establish a baseline with which to compare expression of the key indicators. Statistical methods are used to determine which indicators are needed to reliably monitor disease progression.

Many of these techniques involve large arrays of RNA probes which can monitor expression of thousands of genes at a time, such as the Affymetrix array. Affymetrix makes quartz chips for analysis of DNA Microarrays called GeneChip arrays. Affymetrix's GeneChip arrays assist researchers in quickly scanning for the presence of particular genes in a biological sample. Within this area, Affymetrix is focused on oligonucleotide microarrays. These microarrays are used to determine which genes exist in a sample by detecting specific pieces of mRNA. A single chip can be used to analyze thousands of genes in one assay. However, these systems are expensive to run and their use can be limited in monitoring disease states and treatment efficacy.

RNA for analysis in the present techniques can be derived from a variety of samples, including but not limited to blood, plasma, leukocytes, other blood fractions, sputum, saliva, urine, stool, vaginal swabs, and tissue samples. In various embodiments, cartridges have the capability for automated sample disruption and isolation of RNA. Advantageously, automation of all sample processing improves reliability of RNA isolation without degradation. Furthermore, automation of sample processing enables testing to be run outside of traditional laboratories.

By way of explanation, the method and system described with respect to FIG. 18 provides a system and methods for determining or monitoring the progression of disease states or the efficacy of therapeutic regimens in a subject, preferably a human patient. In particular, the technique relates to methods for monitoring disease states or therapies by monitoring changes in mRNA expression levels. The current technique utilizes a simple, easy to use system which can monitor expression of a number of gene transcripts for rapid diagnosis, to enable better treatment. With respect to the present disclosure, provided is a system and method for RNA profiling to monitor disease state or effectiveness of treatment. The identification of changes in gene expression caused either by the actions of disease states or by therapeutic regimens, such as drug regimens, for disease states is a problem of great commercial and human importance. Most of the decisions that need to be made to run efficient clinical trials and to properly manage the health of patients rely on assays that monitor changes in cells in the body.

The system provides an automated and closed system for isolation of RNA from a patient sample. RNA can be difficult to handle and is susceptible to degradation by RNases. The closed system automates disruption of the sample, cleaning and concentrating of the RNA. During isolation the sample is treated with a guanidine hydrochloride solution, which lyses the cells and disrupts enzymes, thereby stabilizing the RNA. By automating the process in a closed cartridge, the risk of introducing RNases into the sample during handling is eliminated.

The system further provides a multiplexed array to monitor the expression levels of multiple RNA transcripts. As described below, the sensor array has multiple sites specific for individual transcripts. Capture probes specific to a sequence of nucleotides in a transcript are printed at a site. The multiplexed array allows for quantitation of expression of key indicator transcripts and monitoring of housekeeping genes in order to establish baseline expression. The system will then compare expression of the key indicator genes against the housekeeping genes to determine expression relative to the baseline. Variance of key gene expression will be analyzed using an algorithm to determine disease state.

Rather than monitoring all transcripts in a cell, which requires thousands of sensors, the current invention provides an array to monitor between one and hundreds of key transcripts. Additional sensors for one to tens of housekeeping genes would also be included. Optimally the number of key transcripts to be monitored would be between three and twenty. Keeping the number of sites low will minimize the cost of the array but allows for targeted testing of key indicators of a disease.

The current invention has critical functions needed to capture and quantitate RNA expression levels rapidly. In particular, the current invention provides a magnetic approach to concentrate and move RNAs to the sensors to allow for rapid testing without loss of sensitivity. Furthermore, the current system provides a method to quantitate the RNA transcripts at each sensor.

Returning now to FIG. 18, provided is an explanation of how these features may be implemented. For instance, the method 1800 may be performed using a sensor (e.g., sensor system 70 of FIG. 5 or any other example set forth herein) comprising an imager (e.g., imager 72 of FIG. 5 or any other example set forth herein), a flow cell (e.g., flow cell 78 of FIG. 5 or any other example set forth herein) comprising a functionalized surface (e.g., functionalized surface 78 of FIG. 5 or any other example set forth herein) having a plurality of capture probes (e.g., probe molecules 84 of FIG. 5 or any other example set forth herein) coupled to the functionalized surface, a magnet (e.g., magnet 88 of FIG. 5 or any other example set forth herein), and a light source (e.g., light source 126 of FIG. 6 or any other example set forth herein). In the embodiment of FIG. 18, the method starts at block 1801 and includes the following steps:

Step 1810—Binding molecules comprising a first RNA transcript to magnetic particles.

Step 1820—Directing the molecules comprising the first RNA transcript to the functionalized surface via the magnet.

Step 1830—Binding the molecules comprising the first RNA transcript to the capture probe.

Step 1840—Directing a light beam from the light source at the bound molecules comprising the first RNA transcript.

Step 1850—Capturing light from the bound molecules comprising the first RNA transcript.

Step 1860—Determining a quantity of the molecules in the sample comprising the first RNA transcript.

Step 1870—Determining an expression level of the first RNA transcript in the sample based on the quantity of the molecules.

In one embodiment, the method 1800 further includes calculating a disease state or a treatment efficacy based on the plurality of expression levels of the plurality of RNA transcripts. In another embodiment, the plurality of capture probes corresponds to between 10 and 20 RNA transcripts. In a further embodiment, the method further includes monitoring a housekeeping gene for determining a baseline for comparison of the plurality of expression levels of the plurality of RNA transcripts.

In one example, the method further includes receiving and processing the sample without external exposure. In another example, the method further includes binding a plurality of magnetic particles to the molecules and interacting the magnet with the magnetic particles to direct the molecules to the functionalized surface. In another example, the method further includes comprising preventing diffusion of the light beam toward the imager. In a further example, the method further includes binding the molecule to a nanoparticle when the molecule is bound to the functionalized surface, wherein the nanoparticle reflects the light beam toward the imager.

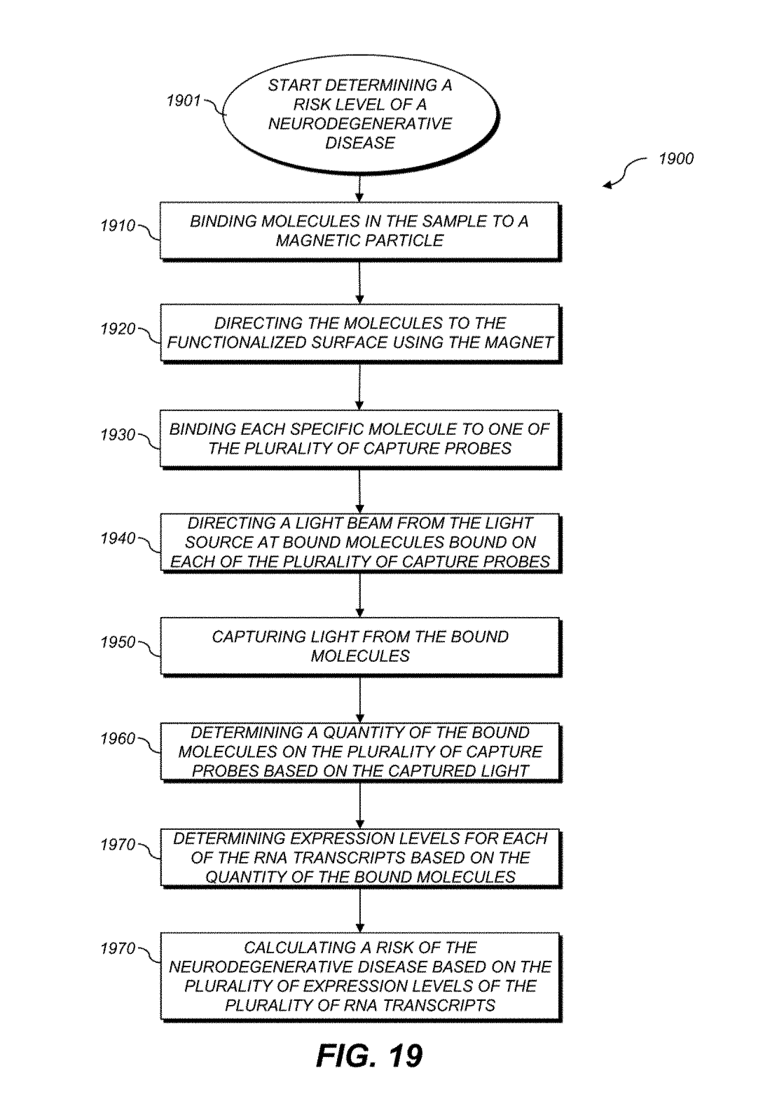

FIG. 19 depicts a method for determining a risk level of a neurodegenerative disease by monitoring expression levels of a plurality of RNA transcripts of a sample.

By way of background, there are two problems in the diagnosis of Alzheimer's disease that would be addressed by the art proposed in this patent application: 1) Accuracy of diagnosis of symptomatic persons and 2) Early detection of disease in persons without symptoms.

Problem 1 is the accuracy of diagnosis. The number of conditions that can cause cognitive deficits that may look like Alzheimer's disease is pages long. Some of these conditions, such as vitamin B deficiency, can be cured, while others may not be curable but might be managed with appropriate intervention. The possibility of cure or potential management require an accurate diagnosis. Unfortunately, research has established that the accuracy of a diagnosis of Alzheimer's disease is poor. If the diagnosis is made by a general practitioner, the probability that the diagnosis is correct is about 50% (Connolly, A., Gaehl, E., Martin, H., Morris, J., Purandare, N., 2011. Under diagnosis of dementia in primary care: variations in the observed prevalence and comparisons to the expected prevalence. Aging Ment. Health 15, 978e984.). If the diagnosis is made in one of the ˜30 federally recognized and funded Alzheimer Centers in the United States, the accuracy is about 75% (Beach, T. G., Monsell, S. E., Phillips, L. E., Kukull, W., 2012. Accuracy of the clinical diagnosis of Alzheimer disease at National Institute on aging Alzheimer disease Centers, 2005-2010. J. Neuropathol. Exp. Neurol. 71, 266e273). The system proposed here would increase the accuracy of diagnosis to better than 90%.

Problem 2 is the early detection of disease. We now know that Alzheimer's disease, Parkinson's disease and many other age-related neurodegenerative diseases start decades before brain damage reaches the point where it is clinically detectable. In the case of Parkinson's disease, 80% of the neurons in the substantia nigra, the region most affected in this disease, are lost before the disease exhibits symptoms that lead to a diagnosis of the disease. Examination for the pathology of Alzheimer's disease in over 3,000 brains of people who died at ages from 10 to 100 showed early Alzheimer pathology in 20% of the brains of people who died in their late 20 s to early thirties (Braak, H., Braak, E., 1997. Frequency of stages of Alzheimer-related lesions in different age categories. Neurobiol. Aging 18, 351e357). It is not until 50 years later that 20% of people are clinically diagnosed with Alzheimer's disease.

This “silent period” for many age-related neurodegenerative diseases creates a window of opportunity for a two-step process. In Step 1 it would be possible to detect disease before significant brain damage has occurred. Step 2 would call for early effective treatment to halt or slow disease progression so that affected persons could live the rest of their lives free of disease symptoms. Free of the shakes and motor losses of Parkinson's disease or free of the memory and cognitive defects of Alzheimer's disease. This application addresses Step 1, accurate diagnosis and early diagnosis.

A variety of methods have been used to show that early diagnosis of Alzheimer's disease is real. Positron Emission Tomography (PET) scanning of the brain in living people has shown Alzheimer pathology in some people as young as the teens or 20 s. Microscopic pathological examination of more than 3,000 brains of people who died between the ages of 10 and 100 has shown the start of Alzheimer's in people in their 20's. And sophisticated cognitive testing has been able to establish indications of Alzheimer's 15 years before the disease became clinically detectable (REF). (Kawas, C. H., Corrada, M. M., Brookmeyer, R., Morrison, A., Resnick, S. M., Zonderman, A. B., Arenberg, D., 2003. Visual memory predicts Alzheimer's disease more than a decade before diagnosis. Neurology 60, 1089e1093.) Studies such as these have been fundamental in establishing that Alzheimer's disease is present in the brain for decades before the brain is damaged to the point of exhibiting frank memory and cognitive problems. However, their cost, or the fact that they rely on postmortem samples, make them prohibitive for detection of disease, or probability of future disease, in the general population.

The present state of the art has several problems. Several technologies have been used to establish the existence of changes in the brain that precede a clinical diagnosis of Alzheimer's disease by many years or decades. Some of these technologies require elaborate equipment and expensive professional expertise. PET imaging is a prime example of this, with the cost of a PET scan of the order of $5,000. This is far too much to be applicable to a population screen, which is what is needed to detect incipient Alzheimer's disease.

Another technology in current use is analysis of protein in spinal fluid, which requires the invasive procedure of a spinal tap performed by a physician. Charges for this procedure can also be of the order of thousands of dollars.

A number of studies have reported that extensive cognitive testing may reveal early, preclinical, signs of Alzheimer's disease. These studies for the most part require extensive time on the part of both patient and tester, also at significant cost.

Recognition of these costs and other drawbacks plus recognition of the need to improve diagnostic accuracy and detection of pre-clinical disease has led to a search for, and descriptions of a blood test for Alzheimer's disease. Some of these are directed a detection of a disease that is already apparent. A few also include an ability to detect preclinical disease. All currently require that samples be sent to a central laboratory for relatively expensive, time consuming procedures. The system proposed here can conduct a test for Alzheimer's disease or other neurodegenerative diseases quickly and on site for minimal cost.

We present here a method for accomplishing the detection of disease or detection of a probable future diagnosis of disease that is low cost and minimally invasive, and, therefore, practical for use on large numbers of persons. Detection of early, incipient disease in the members of a population from perhaps age 30 and beyond requires minimally invasive, inexpensive methods that do not require professional personnel. We here present a method that accomplishes these goals. A method that both provides a more accurate diagnosis of persons presenting with the symptoms that might signify Alzheimer's disease, and that determines a person's risk for a future diagnosis of Alzheimer's disease. The application presented describes methods for obtaining a blood sample, for inserting a sample into an apparatus capable of determining levels of expression of multiple RNA species which have been determined to be useful in multivariate analyses to diagnose and predict Alzheimer's disease. The method described can provide a diagnosis or probability of a future diagnosis in the field within about 30 minutes for a cost we estimate to be less than $100. The system presented is capable of application to screening of a mass population of people without symptoms but who may be at risk of a future diagnosis of Alzheimer's disease.

Returning now to FIG. 19, the method 1900 may be performed using a sensor (e.g., sensor system 70 of FIG. 5 or any other example set forth herein) comprising an imager (e.g., imager 72 of FIG. 5 or any other example set forth herein), a flow cell (e.g., flow cell 78 of FIG. 5 or any other example set forth herein) comprising a functionalized surface (e.g., functionalized surface 78 of FIG. 5 or any other example set forth herein) having a plurality of capture probes (e.g., probe molecules 84 of FIG. 5 or any other example set forth herein) coupled to the functionalized surface, a magnet (e.g., magnet 88 of FIG. 5 or any other example set forth herein), and a light source (e.g., light source 126 of FIG. 6 or any other example set forth herein).

In the embodiment of FIG. 19, the method starts at block 1901 and includes the following steps:

Step 1910—Binding molecules in the sample to a magnetic particle.

Step 1920—Directing the molecules to the functionalized surface using the magnet.

Step 1930—Binding each specific molecule of the molecules to one of the plurality of capture probes configured to bind the RNA transcript of the specific molecule.

Step 1940—Directing a light beam from the light source at bound molecules bound on each of the plurality of capture probes.

Step 1950—Capturing light from the bound molecules.

Step 1960—Determining a quantity of the bound molecules bound on each of the plurality of capture probes based on the captured light.

Step 1970—Determining a plurality of expression levels corresponding to the plurality of RNA transcripts based on the quantity of the bound molecules bound on each of the plurality of capture probes configured to bind each of the plurality of RNA transcript.

Step 1980—Calculating a risk of the neurodegenerative disease based on the plurality of expression levels of the plurality of RNA transcripts.

In one embodiment, the method 1900 further includes calculating the risk of a future diagnosis of the neurodegenerative disease. In another embodiment, the method 1900 further includes calculating the risk of a present diagnosis of the neurodegenerative disease.

In a further embodiment, the method step 1980 of calculating the risk includes using a formula of the form: F=a₀+a₁X₁+a₂X₂+ . . . +aₚXₚ+e, where F is proportional to the risk; p is the number of the plurality of RNA transcripts; Xₙ, for n=1 to p, are the expression levels of each of the plurality of RNA transcripts; aₙ, for n=1 to p, are discriminant coefficients for each of the plurality of RNA transcripts; and e is an error term.

In a specific embodiment, the plurality of RNA transcripts comprise eight DNA transcripts, for n=1 to 8, HSP27, HSP90, GAPDH, FTH, FTL, COX1, COX2, and TFR, and the discriminant coefficients aₙ for the plurality of RNA transcripts comprise, for n=1 to 8, −4.25936, 3.671572, 2.685682, −5.295300, 1.973631, 2.506241, 0.495803, and −1.392785.

In one example, the method 1900 further includes binding a plurality of magnetic particles to the molecules and interacting the magnet with the magnetic particles to direct the molecules to the functionalized surface. In another example, the method 1900 further includes preventing diffusion of the light beam toward the imager. In a further example, the method 1900 further includes binding the molecule to a nanoparticle when the molecule is bound to the functionalized surface, wherein the nanoparticle reflects the light beam toward the imager.

Further implementation details are now set forth in a detailed working example of the present technique.

By way of overview, our basic, laboratory research has established the possibility that quantifying the expression of a number of nucleotide RNA species in a sample and then combining these data yields a number that provides an estimate of a diagnosis of disease or probability of a future diagnosis of disease. In the latter case the measure is of disease biomarkers that are present in sufficient quantity to distinguish from absence of disease, but not yet at a level indicating clinically diagnosable disease. Using the systems and method set forth herein, in one example, we will determine the amount of 10-20 RNA species in each sample The RNA species interrogated will have been selected on the basis of prior and ongoing research. The expression level of each of these genes will then be multiplied by a number. The number will be based on prior and ongoing research and will be different for each RNA species. The multiplier numbers will be the same for the same RNA species in all samples. The resulting 10-20 multiplication products will then be combined into a single number by an algorithm that will indicate the probability of an Alzheimer's disease diagnosis OR the probability of a future diagnosis of Alzheimer's disease.

Details of methods for obtaining blood sample: Blood is drawn from a person for the purpose of determining a diagnosis of or risk of a future diagnosis of a neurodegenerative disease such as Alzheimer's disease, Parkinson's disease, Fronto-temporal dementia, etc. Blood may be drawn from any vein, usually the median cubital vein, and drawn into a syringe. Alternately, blood may be obtained from a finger stick. If drawn from a finger, discard the first drop, then squeeze finger and collect blood onto a Whatman P Card. Let blood dry overnight at room temperature, then store sample at −20 )C. Isolate RNA from the filter paper using QIAGEN kit of your choice—we have used QiAmp and exoRNeasy. If sample is shipped, do so on dry ice. For example, a 2.5 ml or less blood sample could be from a person from any vein with a syringe and needle or similar device. Or, less blood (2-3 drops) could be obtained from a finger stick with a sterile needle or other device to accomplish the finger stick or blood source other than a finger. If from a “finger stick” the blood would be drawn onto filter paper or equivalent. The sample is given an identifying alphanumeric designation that will follow the sample through all further processing. RNA is then extracted from the sample using one of several methods, that may use trizol, PAXgene tubes, phenol/chloroform, etc. As an example, the protocol for blood drawn into a PAXgene tube is as follows.

PAXgene blood RNA Tube Blood Draw protocol:

Ensure that the PAXgene Blood RNA Tube is at 18° C. to 25° C. prior to use and properly labeled with patient ID or code.

If the PAXgene Blood RNA Tube is the only tube to be drawn, blood should be drawn into a “Discard Tube” prior to drawing blood into the PAXgene tube. Otherwise, the PAXgene tube should be the last tube drawn in the phlebotomy procedure.

Collect blood into the PAXgene tube per your institution's recommended procedure for standard venipuncture technique.

Hold the PAXgene Blood RNA Tube vertically, below the blood donor's arm during blood collection.

Allow at least 10 seconds for a complete blood draw to take place. Ensure that the blood has stopped flowing into the tube before removing the tube from the holder.

After blood collection, gently invert the PAXgene tube 8-10 times.

Store the PAXgene tube in an upright position.

Tubes can either be left at room temperature for 2 hours then placed at −20° C. or they can be placed at −20° C. upright in a wire rack immediately after the blood draw.

Storage and Shipping Protocol

To freeze PAXgene tubes, stand them upright in a wire rack. Do not freeze tubes upright in a Styrofoam tray as this may cause the tubes to crack.

For longer term storage at −80° C., tubes must be stored at −20° for at least 24 hours before putting in the −80° C. freezer.

To ship tubes on dry ice, they need to be frozen at −20° C. for at least 24 hours prior to putting on dry ice.

NOTE: Frozen PAXgene Blood RNA Tubes are subject to breakage upon impact. To reduce the risk of breakage during shipment, frozen tubes should be treated in the same manner as glass tubes. It is suggested they be enclosed with bubble wrap or some other kind of treatment for protection. When the PaxGene tube is thawed at room temperature for study, the tube must be inverted at least 6 times to thoroughly mix the contents. Then blood can be withdrawn from the PaxGene tube and inserted directly into a INT supplied cartridge.

An example method for isolating RNA from blood collected into EDTA tubes is as follows: RNA is extracted from leukocytes using the mRNA Isolation Kit for Blood/Bone Marrow (Roche) per manufacturer's protocol. In brief, erythrocytes are selectively lysed and collected by centrifugation. The leukocytes are then lysed and the total nucleic acids is collected by nonspecific adsorption to magnetic beads and magnetic separation. Following a series of washes and elution of the nucleic acids, mRNA is captured by biotin-labeled oligo(dT) and streptavidin-coated magnetic particles. After removal of other nucleic acids (DNA, rRNA, and tRNA) by washing, mRNA samples are collected and stored at −80° C. RNA quality and abundance are confirmed by 260/280 ratios and by gel electrophoresis. Messenger RNA is amplified and radioactively labeled with 32P CTP. The labeled amplified RNA is hybridized to custom cDNA arrays and quantified.

An example method for isolating RNA from blood collected into PAXgene tubes is as follows: Approximately, 2.5 mL of fresh whole blood is collected into PAXgene Blood RNA tubes (BD Diagnostics/Qiagen) and inverted 4 times. Total RNA is extracted from leukocytes with PAXgene Blood RNA Kit (Qiagen) or PAXgene Blood miRNA Kit (Qiagen) per manufacturer's directions. Total RNA is stored at −80° C. until later use. RNA integrity is determined by analysis with an Agilent 2100 Bioanalyzer. The suitability of samples for analysis is based on RNA Integrity Number, with the rare sample with a RIN a number less than 2 considered not usable.

An example method for converting RNA to cDNA is as follows: 1.0 μg of total RNA is reverse transcribed using the High Capacity cDNA Reverse Transcription Kit (Applied Biosystems). The resulting cDNA is diluted 1:5 and used as template in qRT-PCR reactions using TaqMan® Gene Expression Master Mix (Applied Biosystems) and TaqMan® Gene Expression Assays (Applied Biosystems) and run on an iCycler iQ™ (Bio-Rad) or equivalent equipment. The quantitative RT-PCR amplifications are run in triplicate at thermal cycling conditions of 10 minutes at 95° C., 40 cycles of denaturation at 95° C. for 15 seconds, and annealing and extension at 60° C. for 1 minute. Beta glucuronidase (GUSB) is used as an endogenous control since it has been found to be the same from sample to sample. Following normalization, data are then presented as a ratio using the 2(-Delta Delta C(T)) method (Livak and Schmittgen, 2001).

In one embodiment, if blood is obtained from a vein, 2.5 ml is drawn into a PaxGene tube or equivalent following typical protocols, and then the blood is inserted in a cartridge of the present system for internal preparation.

In one embodiment, the following RNA species may be used for calculating risk of neurodegenerative disease. RNA species for study are selected on the basis of known mechanisms of neurodegenerative diseases and on the basis of laboratory findings in preliminary studies. Some examples are shown in the Table 1.

TABLE 1

| Classes of RNA | Examples of class |
|---|---|
| Inflammation | IL-17R, TNF-a, C1 Inhibitor |
| Cell Stress | HSP27, COX2, Alpha1-ACT |
| Epigenetics | HDAC2, DNMT3a, DNM1, |
| Cell Cycle | PCNA, cdc2/cyclin B1, cyclin D, cdk4 |
| Nuclear Transport | NUPL2, NUP155, RAN |
| Protein Folding | IRE1A, BIP, HSC70 |
| Mitochondria | caspasins, COX5a, ATP5B |
| Cell Death | mTOR, p53, |
| RAB system | RAB1, RAB3a, RAB5, RAB 6a, RAB7 |

The weights assigned to each independent variable are corrected for the interrelationships among all the variables. The weights are referred to as discriminant coefficients. The present apparatus quantifies amount of RNA in a sample accurately detect the presence and amount of each of any number of specific RNA or DNA species. In the example provided, 8 specific RNA species are specified, but the identity, specific species and their number may be different. This information about amount of each selected RNA or DNA specie in the sample may be transmitted to an external device such as a cell phone or other mobile computing device. Or, the value may be transmitted to a computing circuit within the same device. The amount detected by the sensor elements for each specie is multiplied in the computing device by a weight that is unique to that RNA or DNA specie. The weight may be referred to as the standardized discriminant coefficient. (Abbreviations: COX1, prostaglandin-endoperoxide synthase 1; COX2, prostaglandin-endoperoxide synthase 2; FTH, ferritin, heavy polypeptide; FTL, ferritin, light polypeptide; GAPDH, glyceraldehyde-3-phosphate dehydrogenase; HSP27, heat shock 27-kDa protein1; HSP90, heat shock protein 90 kDa, class B1; TFR, transferrin receptor).

In this example, this multiplication results in 8 values representing the product of amount of nucleotide specie in the sample multiplied by a weight. The weights may be a product of prior investigation or they may be a result of entering into the system a method for determining weights on the basis of known identity of samples submitted to the system. In the example provided these 8 products of amount of nucleotide multiplied by the weight for each nucleotide are combined by any algorithm that results in a single number that represents a characterization of the sample entered into the system. This number then represents the probability for the detection of a currently diagnosed or probable future diagnosis of Alzheimer's disease or some other neurodegenerative disease, such as Parkinson's disease or amyotrophic lateral stenosis. The resulting number may represent either a diagnosis of a neurodegenerative disease or a probability of a clinical diagnosis of that disease at some time in the future. This information can be presented to any device such as a display in the equipment itself, a local or remote computer or it may wirelessly report data to a secure central site. In the latter case these data can be combined with data from multiple sites to evaluate potential relationships among disease, geography, socio-economic status, etc.

The purpose of discriminant analysis is to obtain a model to predict a single qualitative variable from one or more independent variable(s). Discriminant analysis derives an equation as a combination of the independent variables that will discriminate best between the groups in the dependent variable. This combination is known as the discriminant function. The weights assigned to each independent variable are corrected for the interrelationships among all the variables. The weights are referred to as discriminant coefficients.

The discriminant equation is: F=a₀+a₁X₁+a₂X₂+ . . . +aₚXₚ+e,

where F is formed by the linear combination of the dependent variable, X₁, X₂, . . . Xₚ are the p independent variables, e is the error term and a₀, a₁, a₂, . . . , aₚ are the discriminant coefficients.

Standardized Discriminant Coefficients are calculated for each of the 8 transcripts, as set forth in Table 2.

TABLE 2

| RNA specie | Weight for that RNA |
|---|---|
| HSP27 | −4.25936 |
| HSP90 | 3.671572 |
| GAPDH | 2.685682 |
| FTH | −5.295300 |
| FTL | 1.973631 |
| COX1 | 2.506241 |
| COX2 | 0.495803 |
| TFR | −1.392785 |

By comparison, an example of quantifying amount of specific RNA species in a sample with cDNA or nucleotide arrays is as follows: The cDNA clones represented in arrays emphasized those that would test the hypothesis that transcripts related to stress, inflammation, and cell cycle would be affected in leukocytes from AD cases. 172 cDNAs selected for this purpose were printed on nylon membranes using a 96-pin replicator (Nalge Nunc) with each cDNA spotted in quadruplicate. Arrays are probed with labeled amplified RNA generated from extracted RNA from leukocyte samples and then exposed to a storage phosphor screen. Hybridization intensity of each spot is quantified by laser densitometric scanning (PhosphorImager, Molecular Dynamics). As a control, the amount of cDNA deposited on each spot in the array is quantified by stripping and reprobing the membrane with an oligonucleotide specific for the T7 promoter present in all vectors. Any relevant aspects of this example may be combined with other examples set forth herein.

By comparison, an example of quantifying amount of specific RNA species in a sample by quantitative reverse transcriptase polymerase chain reaction (qPCR) is as follows: The qRT-PCR may be performed using TaqMan Gene Expression Assays (Applied Biosystems, Foster City, Calif., USA). In brief, for each sample, 3.0 μg total RNA is reverse transcribed using a High Capacity cDNA Reverse Transcription Kit (Applied Biosystems). 2 μL of a 1:5 dilution of cDNA is combined with TaqMan Universal PCR Master Mix No AmpErase UNG (Applied Biosystems) and the TaqMan Gene Expression Assay in a 10-μL reaction set-up by the CAS-1200 liquid handling system. The qRT-PCR amplifications may be on an ABI PRISM 7900 HT Sequence Detection System (Applied Biosystems). Universal thermal cycling conditions are: 10 minutes at 95° C., 40 cycles of denaturation at 95° C. for 15 seconds, and annealing and extension at 60° C. for 1 minute. Amplification efficiencies is close to 100% for all assays according to analyses of different dilutions of the cDNA. Beta glucuronidase (GUSB) is used as an endogenous control since its expression is found to be invariant across all samples. Following data normalization, data may be then presented as a ratio using the 2(-Delta C(T)) method (Livak and Schmittgen, 2001). Any relevant aspects of this example may be combined with other examples set forth herein.

An example result of analysis is set forth in Table 3.

TABLE 3

| | | | | | COX1 | |
|---|---|---|---|---|---|---|
| | | DC_{T} | Std. Dev. | Ratio | Relative to | Fold |
| Sample | Status | COX1-GUSB | DC_{T} | COX1/GUSB | Calibrator | Change |
| 514-1 | ND | 1.254108400 | 0.078280830 | 0.419252590 | | |
| 740-1 | Calibrator | 1.695213300 | 0.070636550 | 0.308808999 | | |
| HA01-1 | | 0.993282300 | 0.057280650 | 0.502333606 | | |
| HA02-1 | | 0.442682270 | 0.078432950 | 0.735765395 | | |
| 578 | | −0.153747560 | 0.054353930 | 1.112455448 | | |
| 703-2 | | −0.057216644 | 0.048405107 | 1.040456496 | | |
| 741-1 | | 0.768585200 | 0.044472140 | 0.586992836 | | |
| 1011 | | 0.987758640 | 0.104300660 | 0.504260582 | | |
| | | | | 0.651290744 | 1.00 | 1.00 |
| 561-1 | AD | 1.207212400 | 0.038268127 | 0.433104660 | 0.66 | −1.50 |
| 712-1 | Experimental | 1.099462500 | 0.073005304 | 0.466690337 | 0.72 | −1.40 |
| 765-1 | | 0.940439200 | 0.059617640 | 0.521074226 | 0.80 | −1.25 |
| 775-1 | | 1.561384200 | 0.048603410 | 0.338825838 | 0.52 | −1.92 |
| 661-1 | | 0.901712400 | 0.016683030 | 0.535251040 | 0.82 | −1.22 |
| 698-2 | | 0.969631200 | 0.078005100 | 0.510636582 | 0.78 | −1.28 |
| 772-3 | | 0.473443980 | 0.100681660 | 0.720243190 | 1.11 | 1.11 |
| 811-2 | | 0.240423200 | 0.014296981 | 0.846496965 | 1.30 | 1.30 |

The table above shows the results of a quantitative PCR analysis of COX1 (upper left corner) of 8 non-demented (ND) and 8 Alzheimer cases (AD). Each number under the column “Sample” is the de-identified number given to each case that follows that case through all analyses. The column “Status” indicates whether cases were ND or AD. The column labelled COX1-GUSB is the qPCR result of the strength of signal from qPCR of COX1 for that case in mathematical relation to the invariant signal from the qPCR signal for the enzyme beta glucuronidase (GUSB) which is used as an endogenous control since its expression is invariant across all samples. The next column labelled “std Dev” is the standard deviation of each measure in the preceding column, and is based on the fact that each measure is made in triplicate. The column labelled “Ratio COX1/GUSB” is a ratio mathematical expression of the ratio between expression of COX1 and GUSB for each case. The column “COX1 relative to calibrator” is arbitrarily set at 1 (as is the column labelled “fold change”) for purposes of comparisons with the values from AD cases.

The columns for the AD (Alzheimer disease) cases are similar with the exceptions of the columns labelled labeled “COX1 relative to calibrator” and “fold change” which are now expressions of the relationship between the average value for the ND (non-demented) data and the AD (Alzheimer's disease) data. Appendix I contains all the corresponding tables for each RNA specie in this demonstration. These data are then multiplied by the weights determined for each RNA species.

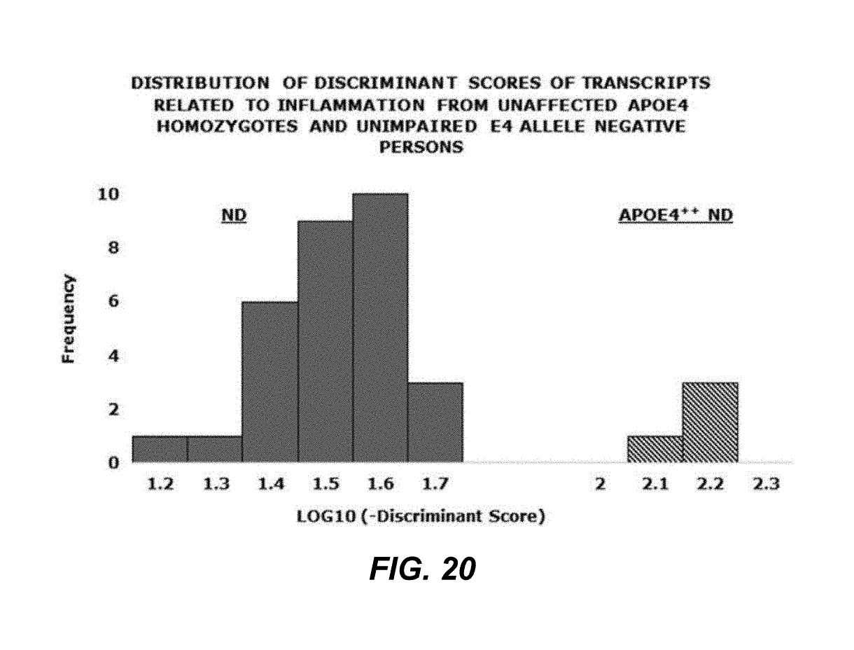

FIG. 20 depicts another working example, for determining risk of a future diagnosis of Alzheimer's disease by virtue of being APOE4++ homozygotes. In this example we are concerned with the risk of a future diagnosis of AD in persons who are presently cognitively intact. Having two copies of the APOE4 gene (one from each parent—APOE4++) constitutes significant risk for a future diagnosis of Alzheimer's disease. The below bar graph showing our blood test scores for people all of whom are cognitively intact. The scores of people who are at increased risk of a future diagnosis of Alzheimer's disease by virtue of having two copies of APOE4 (APOE4++ ND) are clearly separated from the scores of people who do not have the APOE4 gene variant and are not at increased risk of a future diagnosis of Alzheimer's disease

In summary, the discriminant number resulting from our analysis resulting may represent either a diagnosis of a neurodegenerative disease in a symptomatic person or in the case of a normal control person who is cognitively intact it can represent the probability of a clinical diagnosis of that disease at some time in the future. This information can be presented to any device such as a display in the equipment itself, a local or remote computer or it may wirelessly report data to a secure central site. In the latter case these data can be combined with data from multiple sites to evaluate potential relationships among disease, geography, socio-economic status, etc.

Possible advantages of the above described method include improved sensitivity of target molecule detection and improved detection of small quantities of target molecules. In addition, the above described method includes increased speed in detection of target molecules. For example, the above described method can permit detection of target molecules without initial replication of the target molecules, such as in a PCR process.

While the present invention has been particularly shown and described with reference to certain exemplary embodiments, it will be understood by one skilled in the art that various changes in detail may be effected therein without departing from the spirit and scope of the invention that can be supported by the written description and drawings. Further, where exemplary embodiments are described with reference to a certain number of elements it will be understood that the exemplary embodiments can be practiced utilizing either less than or more than the certain number of elements.

The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

To the extent that the claims recite the phrase “at least one of” in reference to a plurality of elements, this recitation is intended to mean at least one or more of the listed elements, and is not limited to at least one of each element. For example, “at least one of an element A, element B, and element C,” is intended to indicate element A alone, or element B alone, or element C alone, or any combination thereof. “At least one of element A, element B, and element C” is not intended to be limited to at least one of an element A, at least one of an element B, and at least one of an element C.

### PARTS LIST

A target molecule binding site
B nanoparticle binding site
X axis
Y axis
10 portable assay system
18 rotor
18P port
20 disposable assay cartridge
21 flow cell
22 cartridge body
22B syringe barrel
24 linear actuator
26 plunger shaft
28 elastomeric plunger
30 central chamber
32 assay chamber
34 assay chamber
40 channel
42 channel
44 bottom panel
50 aperture
70 sensor system
72 imager
74 pixel array
75 lens/objective
76 array circuitry
78 flow cell
80 surface
82 functionalized surface
84 capture probes
86 target molecules
88 magnet
90 method
92-104 method steps
110 magnetic particles
110A magnetic particle
110B magnetic particle
112 magnetic core
114 nanoparticle coating
116 magnetic body
118 movement
120 movement
122 nanoparticles
124 enlarged nanoparticles
126 light source
128 light beam
130 light
132 scattering signature (image)
134 scattering signature (image)
140 optical sensor system
142 imager
144 objective/lens
146 flow cell
147 feeder line
148 light source
150 magnet
152 actuator
160 analysis system
162 base
164 head
166 side surface
167 side surface
168 mirror
170 optical path
180 sensor system
182 prism substrate
184 surface
186 film
188 light source
190 light beam
192 detector
194 light

What is claimed is:

1. A system for analyzing molecules in a sample, the system comprising:
an imager;
a flow cell comprising a functionalized surface comprising a capture probe configured to bind molecules comprising a first RNA transcript;
a magnet positioned opposite the functionalized surface, the magnet configured to direct the molecules comprising the first RNA transcript to the functionalized surface to bind to the capture probe;
a light source configured to direct a light beam at the bound molecules comprising the first RNA transcript, wherein the imager is configured to capture light from the bound molecules comprising the first RNA transcript; and
a processor configured to determine
a quantity of the molecules in the sample comprising the first RNA transcript,
an expression level of the first RNA transcript in the sample based on the quantity of the molecules.

2. The system of claim 1, wherein the system is further configured to calculate a disease state or a treatment efficacy based on the expression level of the first RNA transcript.

3. The system of claim 1, wherein the functionalized surface comprises a second capture probe configured to bind molecules comprising a second RNA transcript, the light source is configured to direct another light beam at the bound molecules comprising the second RNA transcript, the imager is configured to capture light from the bound molecules comprising the second RNA transcript, and the processor is configured to determine a quantity of the molecules in the sample comprising the second RNA transcript.

4. The system of claim 1, wherein the system includes a cartridge for receiving the sample, and the sample is processed within the system without external exposure.

5. The system of claim 1, wherein a plurality of magnetic particles are configured to bind to the molecules in the sample and wherein the magnet is configured to interact with the magnetic particles to direct the molecules to the functionalized surface.

6. The system of claim 1, wherein each of the bound molecules comprising the first RNA transcript is configured to bind a nanoparticle when bound to the functionalized surface and wherein the nanoparticle is configured to reflect the light beam toward the imager.

7. The system of claim 5, wherein the nanoparticle is a gold nanoparticle.

8. The system of claim 5, wherein the nanoparticle is further configured to act as a nucleation site for development of an enlarged nanoparticle.

9. The system of claim 1, further comprising a lens positioned between the imager and the flow cell.

10. A method for analyzing molecules in a sample with an imager, a flow cell comprising a functionalized surface having a capture probe coupled to the functionalized surface, a magnet, and a light source, the method comprising:
binding molecules comprising a first RNA transcript to magnetic particles;
directing the molecules comprising the first RNA transcript to the functionalized surface via the magnet;
binding the molecules comprising the first RNA transcript to the capture probe;
directing a light beam from the light source at the bound molecules comprising the first RNA transcript;
capturing light from the bound molecules comprising the first RNA transcript;
determining a quantity of the molecules in the sample comprising the first RNA transcript; and
determining an expression level of the first RNA transcript in the sample based on the quantity of the molecules.

11. The method of claim 10, further comprising calculating a disease state or a treatment efficacy based on the expression level of the of the first RNA transcript.

12. The method of claim 10, wherein the functionalized surface comprises a second capture probe configured to bind molecules comprising a second RNA transcript, and the method further comprises:
directing another light beam at the bound molecules comprising the second RNA transcript;
capturing light from the bound molecules comprising the second RNA transcript; and
determining a quantity of the molecules in the sample comprising the second RNA transcript.

13. The method of claim 10, further comprising receiving and processing the sample without external exposure.

14. The method of claim 10, further comprising binding a plurality of magnetic particles to the molecules in the sample, and directing the molecules to the functionalized surface with the magnet.

15. The method of claim 10, further comprising preventing diffusion of the light beam toward the imager.

16. The method of claim 10, further comprising binding each of the bound molecules comprising the first RNA transcript to a nanoparticle when bound to the functionalized surface and wherein the nanoparticle is configured to reflect the light beam toward the imager.

17. A method for analyzing molecules in a sample with an imager, a magnet, a light source, and a flow cell comprising a functionalized surface having a plurality of capture probes, each of the plurality of capture probes being configured to bind molecules in the sample comprising one of a plurality of RNA transcripts, the method comprising:
binding molecules in the sample to a magnetic particle;
directing the molecules to the functionalized surface using the magnet;
binding each specific molecule of the molecules to one of the plurality of capture probes configured to bind the RNA transcript of the specific molecule;
directing a light beam from the light source at bound molecules bound on each of the plurality of capture probes;
capturing light from the bound molecules;
determining a quantity of the bound molecules bound on each of the plurality of capture probes based on the captured light; and
determining a plurality of expression levels corresponding to the plurality of RNA transcripts based on the quantity of the bound molecules bound on each of the plurality of capture probes configured to bind each of the plurality of RNA transcripts.
